SEQUENCE LISTING (13 sequences)

SEQ ID NO: 1 - DNA, 28 bp, Artificial Sequence
  Description of Artificial Sequence Synthetic primer
ggggtaccargaccgagaccaccaagac

SEQ ID NO: 2 - DNA, 31 bp, Artificial Sequence
  Description of Artificial Sequence Synthetic primer
gctctagaatcgatgctagccggaggcattg

SEQ ID NO: 3 - DNA, 57 bp, Artificial Sequence
  Description of Artificial Sequence Synthetic oligonucleotide
gatccgtagaaagtgagactcaatttcaagagaattgagtctcactttctacttttt

SEQ ID NO: 4 - DNA, 21 bp, Artificial Sequence
  Description of Artificial Sequence Synthetic oligonucleotide
guagaaagugagacucaautt

SEQ ID NO: 5 - DNA, 21 bp, Artificial Sequence
  Description of Artificial Sequence Synthetic oligonucleotide
auugagucucacuuucuactt

SEQ ID NO: 6 - DNA, 21 bp, Artificial Sequence
  Description of Artificial Sequence Synthetic oligonucleotide
ccuuaggaauagcauuguatt

SEQ ID NO: 7 - DNA, 21 bp, Artificial Sequence
  Description of Artificial Sequence Synthetic oligonucleotide
uacaaugcuauuccuaaggtt

SEQ ID NO: 8 - DNA, 21 bp, Artificial Sequence
  Description of Artificial Sequence Synthetic oligonucleotide
gcucuuucccucaaccuuatt

SEQ ID NO: 9 - DNA, 21 bp, Artificial Sequence
  Description of Artificial Sequence Synthetic oligonucleotide
uaagguugagggaaagagctt

SEQ ID NO: 10 - DNA, 24 bp, Artificial Sequence
  Description of Artificial Sequence Synthetic primer
aacaacgcattgccatagctcgtg

SEQ ID NO: 11 - DNA, 24 bp, Artificial Sequence
  Description of Artificial Sequence Synthetic primer
agtctgcattctggatggtggaca

SEQ ID NO: 12 - DNA, 20 bp, Artificial Sequence
  Description of Artificial Sequence Synthetic primer
agaacatcatccctgcatcc

SEQ ID NO: 13 - DNA, 18 bp, Artificial Sequence
  Description of Artificial Sequence Synthetic primer
agttgctgttgaagtcgc

(19) United States
(12) Patent Application Publication
(10) Pub. No.: US 2013/0253039 A1
(43) Pub. Date: Sep. 26, 2013

(54) COLORECTAL CANCER TREATMENTS AND DIAGNOSTIC IMPROVEMENTS

(71) Applicant: Hiremagalur N. JAYARAM, Indianapolis, IN (US)

(72) Inventor: Hiremagalur N. JAYARAM, Indianapolis, IN (US)

(21) Appl. No.: 13/779,689

(22) Filed: Feb. 27, 2013

Related U.S. Application Data
(60) Provisional application No. 61/603,913, filed on Feb. 27, 2012.

(51) Int. Cl. A61K 31/7088

(52) U.S. Cl. CPC A61K 31/7088
USPC 514/44 R

(57) ABSTRACT

Cancer cells that exhibit low levels of NMNAT are refractory to tiazofurin therapy, and diagnostic methods for assessing NMNAT levels, particularly human NMNAT2, are described, as are compositions and methods for enhancing cytotoxicity towards tiazofurin (2-β-D-ribofuranosylthiazole-4-carboxamide), a pro-drug metabolized by nicotinamide mononucleotide adenylyltransferase (NMNAT) to TAD (thiazole-4-carboxamide adenine dinucleotide). Examples of such compositions include gene delivery vehicles that provide for enhanced NMNAT expression in transfected cells, as well as targeted drug delivery compositions that include tiazofurin encapsulated in folate-tethered nanoparticles. This approach shows that increasing NMNAT levels, particularly hNMNAT2 levels, enhances tiazofurin-mediated cell killing, which has relevance in the treatment of various disease, including various cancers and infectious diseases.

### RELATED APPLICATION

This application claims the benefit of and priority to U.S. provisional patent application Ser. No. 61/603,913, filed 27 Feb. 2012 (attorney docket number: VET-2900-PV), the contents of which is hereby incorporated by reference in its entirety for any and all purposes.

### GOVERNMENT RIGHTS

This work was supported in part by United States Veterans Affairs Merit Review Award (HNJ) entitled, “Targeted Chemo-Gene Therapy for Colorectal Cancer”.

### TECHNICAL FIELD

Improved treatment of colorectal and other cancers by assessing levels of human nicotinamide mononucleotide adenylyltransferase 2 (NMNAT2), followed by treatment with folate-tagged nanoparticles containing a chemotherapeutic agent, are described. Particularly preferred are folate-tagged nanoparticles containing the pro-drug tiazofurin (2-R-D-ribofuranosylthiazole-4-carboxamide), which is metabolized by NMNAT2. Tiazofurin cytotoxicity can be enhanced by increased NMNTA2 expression in colorectal cells.

### BACKGROUND

### 1. Introduction

The following description includes information that may be useful in understanding the present invention. It is not an admission that any of the information provided herein, or any publication specifically or implicitly referenced herein, is prior art, or even particularly relevant, to the presently claimed invention.

### 2. Background

Colorectal cancer is the second most common cause of cancer death in men and women in the United States, with more than 50,000 deaths per year, and each year about 100,000 cases of colon cancer and about 40,000 of rectal cancer are diagnosed in the U.S. 40-50% of patients who undergo potentially curative surgery alone eventually relapse and die of metastatic disease [28]. Standard therapy in metastatic colon cancer, which comprises a chemotherapeutic combination that includes 5-fluorouracil, leucovorin, and oxaliplatin plus anti-vascular endothelial growth factor (VEGF) monoclonal antibody as first line treatment, results in a median survival of 10-15 months [29-31]. Since conventional therapy is relatively non-specific and cytotoxicity occurs in both tumor and normal cells, there is need for selective targeting of colorectal cancer with sparing of normal cells. There is also a need for better understanding whether a particular patient's cancer will respond as intended to a selected course of treatment.

### 3. Definitions

Several terms used in the context of the present invention are defined below. In addition to these terms, others are defined elsewhere in the specification, as necessary. Unless otherwise expressly defined herein, terms of art used in this specification will have their art-recognized meanings.

The term “aberrant” means excessive or unwanted, for example in reference to levels or effective concentrations of a cellular target such as a protein or bioactive lipid.

The term “antibody” (“Ab”) or “immunoglobulin” (Ig) refers to any form of a peptide, polypeptide derived from, modeled after or encoded by, an immunoglobulin gene, or fragment thereof, that is capable of binding an antigen or epitope. See, e.g., Immunobiology, Fifth Edition, C. A. Janeway, P. Travers, M., Walport, M. J. Shlomchiked., ed. Garland Publishing (2001). The term “antibody” is used herein in the broadest sense, and encompasses monoclonal, polyclonal or multispecific antibodies, minibodies, heteroconjugates, diabodies, triabodies, chimeric, antibodies, synthetic antibodies, antibody fragments, and binding agents that employ the complementarity determining regions (CDRs) (or variants thereof that retain antigen binding activity) of the parent antibody. Antibodies are defined herein as retaining at least one desired activity of the parent antibody. Desired activities can include the ability to bind the antigen specifically, the ability to inhibit proleration in vitro, the ability to inhibit angiogenesis in vivo, and the ability to alter cytokine profile(s) in vitro.

Antibodies are usually heterotetrameric glycoproteins of about 150,000 Daltons, typically composed of two identical light (L) chains and two identical heavy (H) chains. Each light chain is typically linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH), also referred to as the variable domain, followed by a number of constant domains. Each light chain has a variable domain at one end (VL) and a constant domain at its other end; the constant domain of the light chain is aligned with the first constant domain of the heavy chain, and the light-chain variable domain is aligned with the variable domain of the heavy chain. Particular amino acid residues form an interface between the light- and heavy-chain variable domains. The terms “variable domain” and “variable region” are used interchangeably. The terms “constant domain” and “constant region” are also interchangeable with each other. Three hypervariable regions (also known as complementarity determining regions or CDRs) in each of the VH and VL regions form the unique antigen binding site of the molecule. Most of the amino acid sequence variation in the antibody molecule is within the CDRs, giving the antibody its specificity for its antigen.

An “antibody derivative” is an immune-derived moiety, i.e., a molecule that is derived from an antibody. This comprehends, for example, antibody variants, antibody fragments, chimeric antibodies, humanized antibodies, multivalent antibodies, antibody conjugates and the like, which retain a desired level of binding activity for antigen.

As used herein, “antibody fragment” refers to a portion of an intact antibody that includes the antigen binding site or variable domains of an intact antibody, wherein the portion can be free of the constant heavy chain domains (e.g., CH2, CH3, and CH4) of the Fc region of the intact antibody. Alternatively, portions of the constant heavy chain domains (e.g., CH2, CH3, and CH4) can be included in the “antibody fragment”. Antibody fragments retain antigen-binding and include Fab, Fab′, F(ab′)2, Fd, and Fv fragments; diabodies; triabodies; single-chain antibody molecules (sc-Fv); minibodies, nanobodies, and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

A “carrier” refers to a moiety adapted for conjugation to a hapten, thereby rendering the hapten immunogenic. A representative, non-limiting class of carriers is proteins, examples of which include albumin, keyhole limpet hemocyanin, hemaglutanin, tetanus, and diptheria toxoid. Other classes and examples of suitable carriers are known in the art. These, as well as later discovered or invented naturally occurring or synthetic carriers, can be adapted for application in accordance with the disclosures herein.

As used herein, the expressions “cell,” “cell line,” and “cell culture” are used interchangeably and all such designations include progeny. Thus, the words “transformants” and “transformed cells” include the primary subject cell and cultures derived there from without regard for the number of transfers. It is also understood that all progeny may not be precisely identical in DNA content, due to deliberate or inadvertent mutations. Mutant progeny that have the same function or biological activity as screened for in the originally transformed cell are included. Where distinct designations are intended, it will be clear from the context.

The term “combination therapy” refers to a therapeutic regimen that involves the provision of at least two distinct therapies to achieve an indicated therapeutic effect. For example, a combination therapy may involve the administration of two or more chemically distinct active ingredients, for example, a fast-acting chemotherapeutic agent and an anti-lipid antibody. Alternatively, a combination therapy may involve the administration of an anti-lipid antibody and/or one or more chemotherapeutic agents, alone or together with the delivery of another treatment, such as radiation therapy and/or surgery. In the context of the administration of two or more chemically distinct active ingredients, it is understood that the active ingredients may be administered as part of the same composition or as different compositions. When administered as separate compositions, the compositions comprising the different active ingredients may be administered at the same or different times, by the same or different routes, using the same of different dosing regimens, all as the particular context requires and as determined by the attending physician. Similarly, when one or more drug species, for example, one or more chemotherapeutic agents are combined with, for example, radiation and/or surgery, the drug(s) may be delivered before or after surgery or radiation treatment.

“Companion diagnostic” refers to a diagnostic test that is linked to a particular drug treatment or therapy. In particular, the diagnostic methods and kits for rapid detection of NMNAT activity or levels in a biological sample, thereby allowing for prompt identification of patients suitable for treatment in accordance with the invention.

The expression “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

To “detect” means to discover or ascertain the existence or presence of (e.g., a disease or condition).

“Diagnosis” means identification of an illness or other condition by examination of its symptoms, including test results and other measurements.

“Effective concentration” refers to the absolute, relative, and/or available concentration and/or activity, for example of certain undesired bioactive lipids. In other words, the effective concentration of a bioactive lipid is the amount of lipid available, and able, to perform its biological function. An immune-derived moiety such as, for example, a monoclonal antibody directed to a bioactive lipid (such as, for example, C1P) is able to reduce the effective concentration of the lipid by binding to the lipid and rendering it unable to perform its biological function. In this example, the lipid itself is still present (it is not degraded by the antibody, in other words) but can no longer bind its receptor or other targets to cause a downstream effect, so “effective concentration” rather than absolute concentration is the appropriate measurement. Methods and assays exist for directly and/or indirectly measuring effective concentrations of bioactive lipids.

An “epitope” or “antigenic determinant” refers to that portion of an antigen that reacts with an antibody antigen-binding portion derived from an antibody.

The term “expression cassette” refers to a nucleotide molecule capable of affecting expression of a structural gene (i.e., a protein coding sequence, such as an antibody chain) in a host compatible with such sequences. Expression cassettes include at least a promoter operably linked with the polypeptide-coding sequence, and, optionally, with other sequences, e.g., transcription termination signals. Additional regulatory elements necessary or helpful in effecting expression may also be used, e.g., enhancers. Thus, expression cassettes include plasmids, expression vectors, recombinant viruses, any form of recombinant “naked DNA” vector, and the like.

To “inhibit,” particularly in the context of a biological phenomenon, means to decrease, suppress or delay. For example, a treatment yielding “inhibition of tumorigenesis” may mean that tumors do not form at all, or that they form more slowly, or are fewer in number than in the untreated control.

An “isolated” composition is one that has been identified and separated and/or recovered from a component of its natural environment. Contaminant components of a natural environment are materials that would interfere with diagnostic or therapeutic uses for the composition, and may include enzymes, hormones, and other proteinaceous or nonproteinaceous solutes. For example, an “isolated” nucleic acid molecule is a nucleic acid molecule that is identified and separated from at least one contaminant nucleic acid molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as it exists in natural cells. However, an isolated nucleic acid molecule includes a nucleic acid molecule contained in cells that ordinarily express the antibody where, for example, the nucleic acid molecule is in a chromosomal location different from that of non-engineered cells.

The word “label” when used herein refers to a detectable compound or composition, such as one that is conjugated directly or indirectly to the antibody. The label may itself be detectable by itself (e.g., radioisotope labels or fluorescent labels) or, in the case of an enzymatic label, may catalyze chemical alteration of a substrate compound or composition that is detectable.

A “liposome” is a small vesicle or micelle composed of various types of lipids, phospholipids, and/or surfactant that is useful for delivery of a drug to a mammal. The components of the liposome are commonly arranged in a bilayer formation, similar to the lipid arrangement of biological membranes.

In the context of this disclosure, a “liquid composition” refers to one that, in its filled and finished form as provided from a manufacturer to an end user (e.g., a doctor or nurse), is a liquid or solution, as opposed to a solid. Here, “solid” refers to compositions that are not liquids or solutions. For example, solids include dried compositions prepared by lyophilization, freeze-drying, precipitation, and similar procedures.

The term “monoclonal antibody” (mAb) as used herein refers to an antibody obtained from a population of substantially homogeneous antibodies, or to said population of antibodies. The individual antibodies comprising the population are essentially identical, except for possible naturally occurring mutations that may be present in minor amounts. Monoclonal antibodies are highly specific, being directed against a single antigenic site. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al., Nature 352:624-628 (1991) and Marks et al., J. Mol. Biol. 222:581-597 (1991), for example, or by other methods known in the art. The monoclonal antibodies herein specifically include chimeric antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)).

“Monotherapy” refers to a treatment regimen based on the delivery of one therapeutically effective compound, whether administered as a single dose or several doses over time.

“Neoplasia” or “cancer” refers to abnormal and uncontrolled cell growth. A “neoplasm”, or tumor or cancer, is an abnormal, unregulated, and disorganized proliferation of cell growth, and is generally referred to as cancer. A neoplasm may be benign or malignant. A neoplasm is malignant, or cancerous, if it has properties of destructive growth, invasiveness, and metastasis. Invasiveness refers to the local spread of a neoplasm by infiltration or destruction of surrounding tissue, typically breaking through the basal laminas that define the boundaries of the tissues, thereby often entering the body's circulatory system. Metastasis typically refers to the dissemination of tumor cells by lymphatics or blood vessels. Metastasis also refers to the migration of tumor cells by direct extension through serous cavities, or subarachnoid or other spaces. Through the process of metastasis, tumor cell migration to other areas of the body establishes neoplasms in areas away from the site of initial appearance.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

A “patentable” composition, process, machine, or article of manufacture means that the subject matter satisfies all statutory requirements for patentability at the time the analysis is performed. For example, with regard to novelty, non-obviousness, or the like, if later investigation reveals that one or more claims encompass one or more embodiments that would negate novelty, non-obviousness, etc., the claim(s), being limited by definition to “patentable” embodiments, specifically exclude the non-patentable embodiment(s). Also, the claims appended hereto are to be interpreted both to provide the broadest reasonable scope, as well as to preserve their validity. Furthermore, the claims are to be interpreted in a way that (1) preserves their validity and (2) provides the broadest reasonable interpretation under the circumstances, if one or more of the statutory requirements for patentability are amended or if the standards change for assessing whether a particular statutory requirement for patentability is satisfied from the time this application is filed or issues as a patent to a time the validity of one or more of the appended claims is questioned.

The term “pharmaceutically acceptable salt” refers to a salt, such as used in formulation, which retains the biological effectiveness and properties of the agents and compounds described herein and which are is biologically or otherwise desirable. In many cases, the agents and compounds described herein are capable of forming acid and/or base salts by virtue of the presence of charged groups, for example, charged amino and/or carboxyl groups or groups similar thereto. Pharmaceutically acceptable acid addition salts may be prepared from inorganic and organic acids, while pharmaceutically acceptable base addition salts can be prepared from inorganic and organic bases. For a review of pharmaceutically acceptable salts (see Berge, et al. (1977) J. Pharm. Sci., vol. 66, 1-19).

A “plurality” means more than one.

“Point-of-care testing” means medical testing or diagnosis at or near the site of patient care.

The term “promoter” includes all sequences capable of driving transcription of a coding sequence in a cell. Thus, promoters used in the constructs described herein include cis-acting transcriptional control elements and regulatory sequences that are involved in regulating or modulating the timing and/or rate of transcription of a gene. For example, a promoter can be a cis-acting transcriptional control element, including an enhancer, a promoter, a transcription terminator, an origin of replication, a chromosomal integration sequence, 5′ and 3′ untranslated regions, or an intronic sequence, which are involved in transcriptional regulation. Transcriptional regulatory regions suitable for use include but are not limited to the human cytomegalovirus (CMV) immediate-early enhancer/promoter, the SV40 early enhancer/promoter, the E. coli lac or trp promoters, and other promoters known to control expression of genes in prokaryotic or eukaryotic cells or their viruses. The term “recombinant DNA” refers to nucleic acids and gene products expressed therefrom that have been engineered, created, or modified by man. “Recombinant” polypeptides or proteins are polypeptides or proteins produced by recombinant DNA techniques, for example, from cells transformed by an exogenous DNA construct encoding the desired polypeptide or protein. “Synthetic” polypeptides or proteins are those prepared by chemical synthesis.

The terms “separated”, “purified”, “isolated”, and the like mean that one or more components of a sample contained in a sample-holding vessel are or have been physically removed from, or diluted in the presence of, one or more other sample components present in the vessel. Sample components that may be removed or diluted during a separating or purifying step include, chemical reaction products, non-reacted chemicals, proteins, carbohydrates, lipids, and unbound molecules.

By “solid phase” is meant a non-aqueous matrix such as one to which an antibody can adhere directly or indirectly. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes beads or a discontinuous solid phase of discrete particles, such as those described in U.S. Pat. No. 4,275,149.

The term “species” is used herein in various contexts, e.g., a particular species of chemotherapeutic agent. In each context, the term refers to a population of chemically indistinct molecules of the sort referred in the particular context.

A “subject” or “patient” refers to an animal to which treatment is given. Animals that can be treated include vertebrates, with mammals such as bovine, canine, equine, feline, ovine, porcine, and primate (including humans and non-human primates) animals being particularly preferred examples.

A “therapeutic agent” refers to a drug or compound that is intended to provide a therapeutic effect including, but not limited to tiazofurin.

A “therapeutically effective amount” (or “effective amount”) refers to an amount of an active ingredient, e.g., a composition of the invention, sufficient to effect treatment when administered to a subject in need of such treatment. Accordingly, what constitutes a therapeutically effective amount of a composition may be readily determined by one of ordinary skill in the art. The therapeutically effective amount will depend upon the particular subject and condition being treated, the weight and age of the subject, the severity of the disease condition, the particular compound chosen, the dosing regimen to be followed, timing of administration, the manner of administration and the like, all of which can readily be determined by one of ordinary skill in the art. It will be appreciated that in the context of combination therapy, what constitutes a therapeutically effective amount of a particular active ingredient may differ from what constitutes a therapeutically effective amount of the active ingredient when administered as a monotherapy (i.e., a therapeutic regimen that employs only one chemical entity as the active ingredient).

The compositions described herein are used in methods of gene-directed enzyme pro-drug therapy. As used herein, the terms “therapy” and “therapeutic” encompasses the full spectrum of prevention and/or treatments for a disease, disorder or physical trauma. A “therapeutic” agent may act in a manner that is prophylactic or preventive, including those that incorporate procedures designed to target individuals that can be identified as being at risk (pharmacogenetics); or in a manner that is ameliorative or curative in nature; or may act to slow the rate or extent of the progression of at least one symptom of a disease or disorder being treated; or may act to minimize the time required, the occurrence or extent of any discomfort or pain, or physical limitations associated with recuperation from a disease, disorder, or physical trauma; or may be used as an adjuvant to other therapies and treatments.

The term “treatment” or “treating” means any treatment of a disease or disorder, including preventing or protecting against the disease or disorder (that is, causing the clinical symptoms not to develop); inhibiting the disease or disorder (i.e., arresting, delaying or suppressing the development of clinical symptoms; and/or relieving the disease or disorder (i.e., causing the regression of clinical symptoms). As will be appreciated, it is not always possible to distinguish between “preventing” and “suppressing” a disease or disorder because the ultimate inductive event or events may be unknown or latent. Those “in need of treatment” include those already with the disorder as well as those in which the disorder is to be prevented. Accordingly, the term “prophylaxis” will be understood to constitute a type of “treatment” that encompasses both “preventing” and “suppressing”. The term “protection” thus includes “prophylaxis”.

The term “therapeutic regimen” means any treatment of a disease or disorder using chemotherapeutic and cytotoxic agents, radiation therapy, surgery, gene therapy, DNA vaccines and therapy, siRNA therapy, anti-angiogenic therapy, immunotherapy, bone marrow transplants, aptamers and other biologics such as antibodies and antibody variants, receptor decoys and other protein-based therapeutics.

A “vector” or “plasmid” or “expression vector” refers to a nucleic acid that can be maintained transiently or stably in a cell to effect expression of one or more recombinant genes. A vector can comprise nucleic acid, alone or complexed with other compounds, and may be a viral or non-viral vector. A vector optionally comprises viral or bacterial nucleic acids and/or proteins, and/or membranes. Vectors include, but are not limited, to replicons (e.g., RNA replicons, bacteriophages) to which fragments of DNA may be attached and become replicated. Thus, vectors include, but are not limited to, RNA, autonomous self-replicating circular or linear DNA or RNA and include both the expression and non-expression plasmids. Plasmids can be commercially available, publicly available on an unrestricted basis, or can be constructed from available plasmids as reported with published protocols. In addition, the expression vectors may also contain a gene to provide a phenotypic trait for selection of transformed host cells such as dihydrofolate reductase or neomycin resistance for eukaryotic cell culture, or such as tetracycline or ampicillin resistance in E. coli.

### SUMMARY

This invention addresses several shortcomings of current treatments for colorectal and other cancers. One object of the invention concerns determining whether a patient's cancer is likely to respond to the pro-drug tiazofurin, which is metabolized by hNMNAT, particularly isoform 2 (hNMNAT2). Thus, one aspect of the invention concerns determining a level of hNMNAT2 expression in cells obtained from a patient. NMTAT2 expression can be assessed at the level of messenger RNA or protein by any suitable method, alone or in conjunction with one or more other biomarkers.

If hNMNAT2 expression is below a threshold level, the patient may be treated by a combination that involves enhancing hNMNAT2 expression and providing a pro-drug metabolized by NMNAT2 (e.g., tiazofurin), which is another object of the invention. NMNAT2 expression can be increased by any suitable method, including by delivery of a expression construct that codes for production of hNMNAT2 or that enhances expression of endogenous hNMNAT2, or by delivery of hNMNAT2 protein. The component that enhances hNMNAT2 expression can be delivered separately from the pro-drug or in conjunction with the desired chemotherapeutic agent, as part of the same or different compositions. If administered separately, the compositions may be delivered at the same or different times, by the same or different routes, and in accordance with the same or different dosing regimens.

Yet another object concerns cell-targeted cancer therapy. In one aspect, folate receptors (FR) are targets for folate-tagged nanoparticles carrying a chemotherapeutic payload, for example, tiazofurin. A particularly preferred folate-tagged (or tethered) nanoparticle composition comprises distearoylphosphatidylcholine (DSPC), cholesterol, and DSPE-PEG-folate (56:40:0.1 v/v), which can then be loaded with a desired amount of a selected chemotherapeutic agent such as tiazofurin.

These and other aspects and embodiments of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### BRIEF DESCRIPTION OF THE FIGURES

A brief summary of each of the figures referenced in this specification appears below. This application contains at least one figure executed in color. Copies of this application with color drawings will be provided upon request and payment of the necessary fee.

FIG. 1: Schematic Representation of Tiazofurin Activation to TAD and Inhibition of Guanylate Synthesis.

Tiazofurin is activated to form TAD an analogue of NAD by NMNAT. TAD competes with NAD, a cofactor for IMPDH and thus inhibits guanylate synthesis. (TR-tiazofurin; TRMP— tiazofurin 5′-monophosphate; TAD-thiazole-4-carboxamide adenine dinucleotide; NMNAT-nicotinamide mononucleotide adenylyl transferase; NMN—nicotinamide 5′-monophosphate; NAD—nicotinamide adenine dinucleotide; IMP—inosine 5′-monophosphate; IMPDH—inosine 5′-monophosphate dehydrogenase; XMP—xanthosine 5′-monophosphate; GTP—guanosine triphosphate; dGTP—deoxyguanosine triphosphate).

FIG. 2: Relationship of hNMNAT2 Expression with Tiazofurin Cytotoxicity in Human Colorectal Cancer Cell Lines.

Cells were processed as detailed in the Methods section below. Panel A, hNMNAT2 expression in colorectal cancer cell lines and bar graph of hNMNAT2 expression in relation to GAPDH expression as a percent of HCT15; Panel B, cytotoxicity of colorectal cancer cell lines to tiazofurin; Panel C, regression analysis of relationship between hNMNAT2 expression and cytotoxicity to tiazofurin; Panels D and F, FACS analysis of GFP fluorescence in wild-type and hNMNAT2 transfected cell lines (—wild-type; hNMNAT2 transfected); Panels E and G, Western blot analyses of hNMNAT2 expression in wild-type (WT) and hNMNAT2 transfected cell lines.

FIG. 3: Influence of Transfection of hNMNAT2 in Caco2 Cell Lines.

Panel A, Cells were Processed and NAD levels were determined in the cytoplasmic extracts as described in the Methods section, below. The y-axis of the bar graph shows the nanomoles (nmol) of NAD concentration for million cells; Panel B, IMPDH2 expression in wild-type and hNMNAT2 transfected Caco2 cell lines and the bar graph of IMPDH2 expression in relation to GAPDH expression as a percent of wild-type; Panel C, IMPDH activity in wild-type and hNMNAT2 transfected Caco2 cell lines and the influence of tiazofurin on their activity. Triplicate flasks were set-up with each cell line and incubated with saline or tiazofurin (100-300 μM) for 4 h at 3TC. Cells were processed as described in the Methods section below. Basal IMPDH activity in saline treated control was 1.85 pmol (micromole) of XMP synthesized/mg protein/min; Panel D, NMNAT enzyme activity in wild-type and hNMNAT2 transfected Caco2 cell lines. The y-axis of the bar graph indicates the NMNAT activity in unit/mg of protein; Panel E, hNMNAT2 expression in cytoplasm, nucleus, and mitochondria. Cytoplasmic, nuclear, and mitochondrial proteins were isolated as detailed in the Methods section below and expression of hNMNAT2 protein was determined by Western blot analysis in wild-type and hNMNAT2 transfected caco2 cell lines; Panel F, silencing of hNMNAT2 expression in hNMNAT2 overexpressing cell line. hNMNAT2 expression in transfected Caco2 cell lines was silenced by shRNA-NMNAT2 and there expression was determined by Western blot analysis; Panel G, the wild-type, hNMNAT2 overexpressing, and hNMNAT2 overexpressing Caco2 cell lines were subjected to cell-kill by tiazofurin. [symbol] Wild-type (WT), [symbol] hNMNAT2 transfected cell line, [symbol] shRNA-hNMNAT2 cell line.
*Denotes statistical significance (P<0.05) NS Denotes not significant.

FIG. 4: Effect of GTP and TAD Concentration Following Tiazofurin Treatment.

Panels A, B, D, and E, triplicate flasks were set-up with each cell line, treated with tiazofurin (0, 150 and 300 μM) for Caco2 and (0, 50 and 150 μM) for HT29 for 4 h and the cells were processed as detailed in Methods section below. The y-axis of the bar graph indicate the GTP concentration in nmol per million cells while the x-axis gives the tiazofurin concentration used; Panels C and F, effect of TAD concentration following tiazofurin treatment. Triplicate flasks were set-up with each cell line, treated with tiazofurin Caco2 (300 μM), and HT29 (150 μM) for 4 h and processed as detailed in Methods section below. The y-axis indicates the TAD concentration in nmol per million cells. [symbol] Wild-type (WT), [symbol] hNMNAT2 transfected cell line.
*Denotes statistical significance (P<0.05) NS Denotes not significant

FIG. 5: Effect of Tiazofurin Encapsulated in Folate-Tethered and Non-Targeted Nanoparticles on Cell-Kill in Caco2 and HT29 Cell Lines.

Panels A and D, FACS analysis of calcein uptake. Caco2 and HT29 cell lines where treated with folate-tethered and non-targeted nanoparticles and calcein uptake are a measure of Folate receptor targeting. The vertical abscissa represents number of relative value of fluorescence in cells counted and the coordinate abscissa indicates cell lines; Panels B, C, E and F, Caco2 and HT29 cell lines were grown for at least 20 generations in low-folate medium before being used for determining FR expression and cell-kill by tiazofurin. Folate-tethered and non-targeted nanoparticles encapsulated with tiazofurin were prepared as detailed in the Methods section below. The bar graph Caco2 (B and C) and HT29 (E and F) were show the difference in cell-kill on exposure to different treatments. [symbol] Free tiazofurin, [symbol] tiazofurin encapsulated in non-targeted nanoparticles, [symbol] tiazofurin encapsulated in folate-targeted nanoparticles.
*Denotes statistical significance (P<0.05) NS Denotes not significant.

FIG. 6: hNMNAT1 Silencing on Cell-Kill by Tiazofurin.

Panels A and D, cells were processed as detailed in the Methods sertion below. Western blot analysis of hNMNAT1 in Caco2 and HT29 (wild-type and hNMNAT2 transfected) cell lines as a function of days following the transfection of siRNA against NMNAT1. The results are representative from three independent sets of experiments. The table below each cell line shows the maximum reduction in mRNA level of NMNAT1 corresponding to cytotoxicity at that time point. Panels B and D show the influence of siRNA-hNMNAT1 treatment on day 3 on tiazofurin cytotoxicity in Caco2 and HT29 cell lines.

FIG. 7: hNMNAT3 Silencing on mRNA Expression and Cell-Kill by Tiazofurin.

Quantitative real-time RT-PCR (qRT-PCR) determination of hNMNAT3 mRNA levels in Caco2 (A and B) and HT29 (D and E) (wild-type and hNMNAT2 overexpressing) cell lines as a function of days following the transfection of siRNA against NMNAT3 mRNA. The data using siRNA control as open bars is expressed as the 100 percent value and the data on NMNAT3 mRNA is shown in closed bars is expressed as a percent of control values. The result is presented as the mean±S.D. from three independent sets of experiments with each data point performed in triplicate. The table below each cell line shows the maximum reduction in mRNA level of NMANT3 corresponding to cytotoxicity at that time point. Panels C and F show the influence of siRNA-hNMNAT3 treatment after 24 h on tiazofurin cytotoxicity in Caco2 and HT29 cell lines.

FIG. 8: hNMNAT2 Expression in Wild-Type and hNMNAT2-Over-Expressed HT29 Cell Line, In Vitro and In Vivo.

hNMNAT2 expression levels in vitro and in vivo of wild-type and hNMNAT2-overexpressing HT29 cells transplanted into athymic mice.

FIG. 9: The Effect of Tiazofurin on Wild-Type and hNMNAT2-Over-Expressed HT29 Tumors, In Vivo.

### DETAILED DESCRIPTION

### Overview

Colorectal cancer cells exhibit limited cytotoxicity towards tiazofurin (2-R-D-ribofuranosylthiazole-4-carboxamide), a pro-drug metabolized by nicotinamide mononucleotide adenylyltransferase (NMNAT) to TAD (thiazole-4-carboxamide adenine dinucleotide), a potent inhibitor of inosine 5′-monophosphate dehydrogenase (IMPDH). IMPDH is the rate-limiting enzyme for de novo purine synthesis, and inhibition of IMPDH results in reduced levels of cellular guanylates their synthesis, resulting in the inhibition tumor cell growth in vitro and in vivo. Here, it is shown that colorectal cancer cell lines that exhibit low levels of NMNAT are refractory to tiazofurin therapy but can be rendered sensitive to tiazofurin by NMNAT overexpression. These results also demonstrate a strong correlation between expression of human NMNAT2 (hNMNAT2) and tiazofurin induced cell-kill in cancer cell lines, and transfection of hNMNAT2 resulted in a 6- and 2-fold cytoplasmic overexpression in Caco2 and HT29 cell lines, respectively. Furthermore, to achieve targeted drug delivery, tiazofurin was encapsulated in folate-tethered nanoparticles, and the results demonstrated increased selective cell-kill of cells that overexpressed hNMNAT2. Tiazofurin sensitivity was not altered by silencing either hNMNAT1 or hNMNAT3 expression. Folate receptors (FR), which are overexpressed in more than 90% of colorectal carcinomas, can be exploited for cell-specific targeting, for example, with tiazofurin encapsulated in folate-tethered nanoparticles for gene targeted enzyme pro-drug therapy. These results demonstrate that colorectal and other cancer cell killing by tiazofurin can be enhanced by overexpressing hNMNAT2 and further targeting of cell surface FR by folate-tethered nanoparticles increased tiazofurin cell-kill. This new inventive treatment approach shows that that gene (hNMNAT2) targeted enzyme-pro-drug (Tiazofurin) therapy enhances tiazofurin cell-kill.

### Detailed Description

1. NMNAT and Tiazofurin

Nicotinamide adenine dinucleotide (NAD⁺) is a high-energy molecule that can be reduced to form NADH in various cellular processes. The NAD⁺/NADH ratio plays an important role in intracellular redox state and is a good index of the metabolic state of the cell. NAD⁺ is synthesized by the action of NMNAT utilizing NMN and ATP as substrates to form NAD⁺ and PPi. Although NAD⁺ can be produced either by conserved de novo or salvage pathways, both pathways are mediated by NMNAT, the crucial rate-limiting step to catalyze the final product NAD⁺. Three human nicotinamide 5′-mononucleotide adenylyltransferase (hNMNAT) genes have been discovered. The human NMNAT1 gene exhibits nuclear localization signal and is found in the nucleus [1]. Human NMNAT2 was independently cloned and found to be present in the cytoplasm and localized in Golgi bodies [2-4]. Human NMNAT3 was cloned and shown to be present in the mitochondria [4].

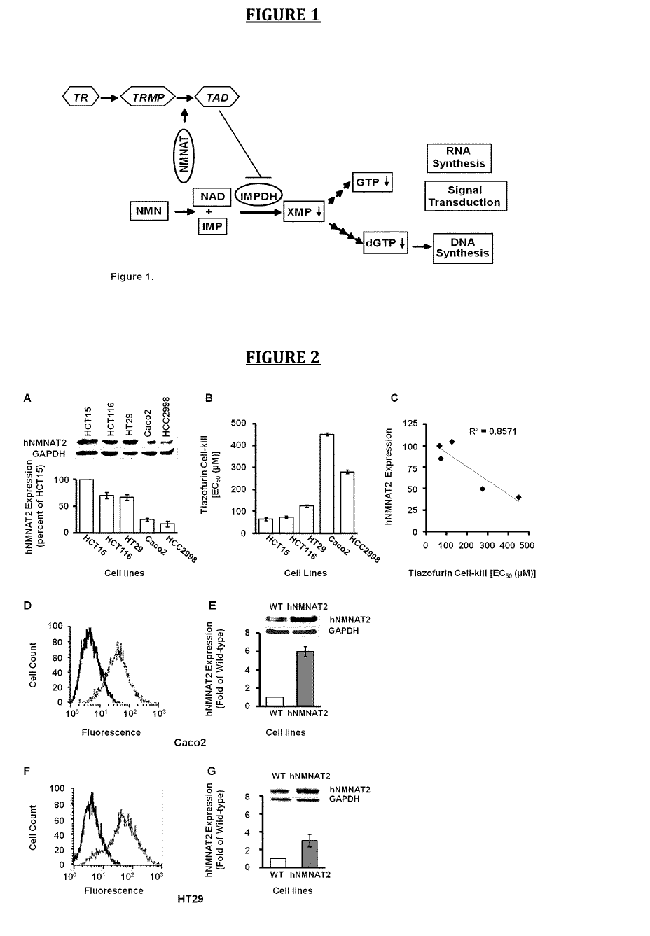

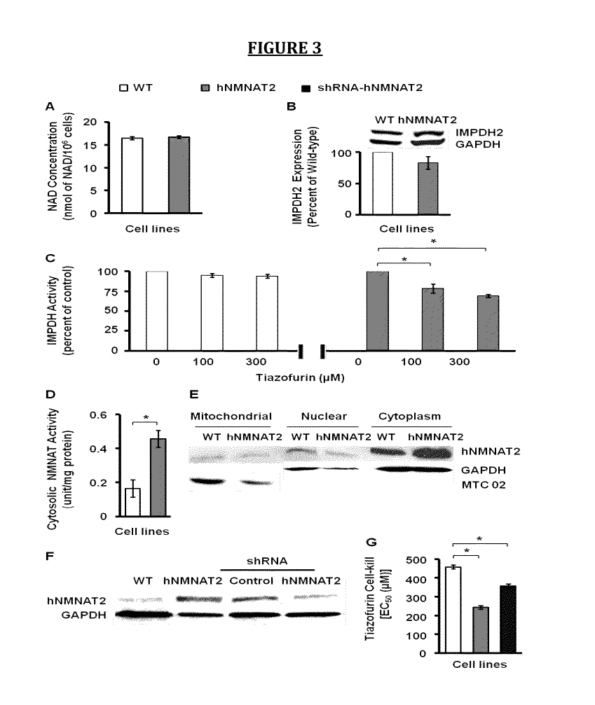

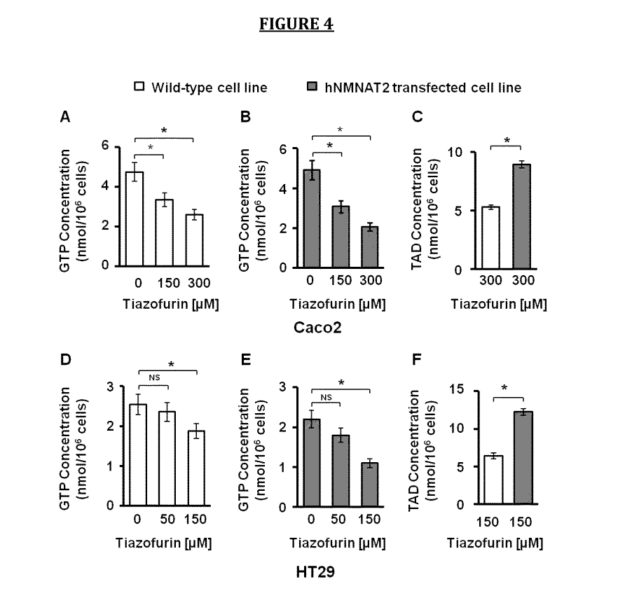

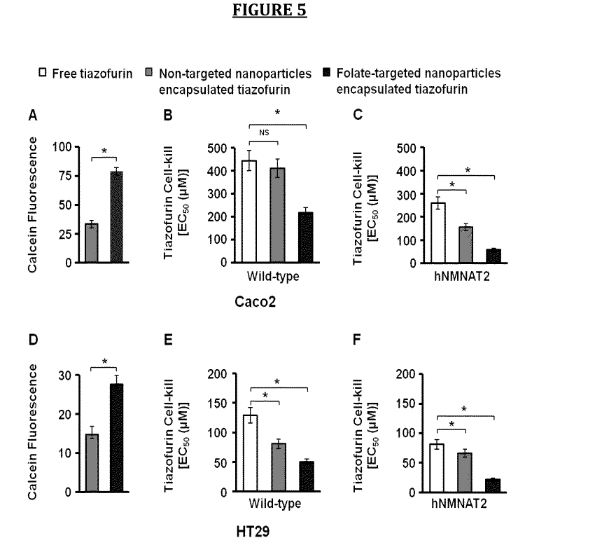

Tiazofurin is a nucleoside pro-drug which is phosphorylated to tiazofurin 5-monophosphate and then converted in sensitive cells by the action of NMNAT, to its active metabolite, thiazole-4-carboxamide adenine dinucleotide (TAD), an analog of NAD⁺ [5]. TAD inhibits inosine 5′-monophosphate dehydrogenase (IMPDH) which is a rate-limiting enzyme in the synthesis of guanylates, including GTP and dGTP (FIG. 1). Sensitivity to tiazofurin was shown to be related to the cellular NMNAT activity and tiazofurin-resistant cell lines showed decreased NMNAT activity [6]. Tiazofurin exhibits decreased cytotoxicity to human colorectal cancer cells compared to leukemic cell lines in vitro and requires high doses of tiazofurin to exhibit in vivo antitumor activity in mice transplanted subcutaneously with colorectal cancer HT-29 [7].

Depletion of GTP pools can strongly interfere with G-protein-mediated signal transduction, thereby inducing phenotypic changes in gene expression. Therefore, using drugs to reduce GTP in cells has proven to be very useful in clinical therapeutics since reduction in GTP results in eventual cancer cell death [8-10]. Tiazofurin demonstrated a true specific reduction in GTP, which correlated with evidence of response in Phase I/II clinical trials in patients with chronic myeloid leukemia in blast crisis [9,10]. As a result of its good activity in phase II clinical trials in leukemic patients, tiazofurin was approved as an orphan drug for the treatment of patients with chronic myelogenous leukemia in blast crisis [11]. However, in a Phase II study with tiazofurin in 21 patients with colorectal cancer, there was no significant antitumor activity [12]. The basis for its lack of significant antitumor activity in early phase II studies with colorectal cancer patients may be related to several factors: first, the level of NMNAT was not determined in the study and may be very low in colorectal tumors of patients; second, tiazofurin was administered as a bolus (within 10 minutes) once a day for 5 days, and later studies in patients with chronic myeloid leukemia in blast crisis showed that tiazofurin needs to be administered over longer periods (10 days) to effectively inhibit IMPDH to reduce GTP levels and to induce antitumor effect [9,10]; and third, pharmacokinetic studies in patients treated with tiazofurin demonstrated that sufficient plasma tiazofurin levels were not reached in tumor tissue after low-dose bolus treatment [13].

Subsequent studies with tiazofurin demonstrated significant antitumor activity in a liver metastasis athymic mouse model of human colorectal carcinoma [7,14]. Tiazofurin inhibited metastasis formation in mouse liver and reduced dose-dependent micro-invasiveness of colon cancer cells, in vivo [14]. Earlier studies with murine tumors spontaneously sensitive and resistant to tiazofurin indicated that tumors exhibiting high NMNAT activity showed exquisite sensitivity to tiazofurin [15]. Moreover, tumors that were sensitive to tiazofurin also exhibited high TAD levels with concurrent decrease in GTP concentration, demonstrating that tumors that exhibited high NMNAT activity avidly metabolized tiazofurin to TAD [8,15,16].

This invention is based on the observation that certain cancer cell lines that exhibit low levels of NMNAT are refractory to tiazofurin therapy but can be rendered sensitive to tiazofurin by NMNAT overexpression. Thus, some aspects of the invention concern methods and kits detecting or identifying diseased cells that exhibit low levels of NMNAT activity. Another aspect of the invention relates to rendering such cells sensitive to tiazofurin by NMNAT overexpression. Such sensitized cells can then be effectively treated by administering compositions that comprise tiazofurin.

NMNAT activity or levels (particularly hNMNAT2 activity or levels) can be assessed by any suitable method, including enzymatic assays, physical measurements (e.g., mass spectrometry, LC-MS), and methods which rely on specific hNMNAT2-binding agents such as antibodies to hNMNAT2, hNMNAT2-binding antibody fragments, and the like. Antibody-based methods such as ELISA or other immunochemical assays for detecting hNMNAT2 levels are particularly preferred, and can be used in clinical or point of care diagnostic assay formats.

The determining of hNMNAT2 activity or levels may be performed by any suitable now-known or later developed method, for example, a method based on an hNMNAT2-binding agent, or reagent such as an antibody-based method, e.g., enzyme-linked immunosorbent assay (ELISA), lateral flow immunoassay (LFIA), or immunohistochemistry (1HC); a physical measurement method, e.g., mass spectrometry or liquid chromatography/mass spectrometry; or a method to assess NMNAT enzymatic activity. The anti-NMNAT antibody or NMNAT-binding antibody fragment used in such an antibody-based method is an antibody or antigen-binding that specifically binds NMNAT. The method may further comprise determining levels of at least one additional biomarker for the cancer to be treated in the biological sample, such as a lipid, protein, peptide, or nucleic acid biomarker. Such methods of detection typically comprise determining NMNAT activity or levels in a biological sample from the subject. Biological samples include tissue samples (e.g., from biopsies) or a bodily fluid sample, e.g., a sample of cerebrospinal fluid (CSF), blood, plasma, urine, or other bodily fluid (or traction thereof).

Further envisioned by the invention are kits for detecting or diagnosing hNMNAT2 activity or levels in a subject. Such kits typically include components for determining hNMNAT2 activity or levels in a biological sample from a subject, wherein decreased hNMNAT2 activity or levels in the sample (e.g., from a tumor biopsy) indicates lack of sensitivity to tiazofurin. Administration of a gene delivery vehicle, preferably via targeted delivery, coding for expression of hNMNAT2, or hNMNAT2 protein, would then sensitize such cells to tiazofurin treatment. Kits include those for performing ELISAs, lateral flow immunoassays (LFIAs), or immunohistochemistry (1HC), physical measurements (e.g., mass spectrometry or liquid chromatography/mass spectrometry), or NMNAT enzymatic activity assays. For example, a kit for performing a lateral flow immunoassay may comprise an anti-NMNAT antibody or antigen-binding fragment of an anti-NMNAT monoclonal or polyclonal antibody that is directly or indirectly bound to a solid support (e.g., a lateral flow chip or strip) or a carrier moiety, e.g., polyethylene glycol, colloidal gold, adjuvant, a silicone bead, a latex bead, or a protein e.g., keyhole limpet hemocyanin, albumin, bovine thyroglobulin, or other carriers and supports known in the art. The solid support may also contain reagents for detecting other biomarkers and one or more controls. In some embodiments, the carrier is colored or carries a detectable label.

In some embodiments, such kits also include reagents for detecting bound NMNAT, which may be secondary antibodies or antigen-binding antibody fragments that carry a detectable label.

Detectable labels are available which can be generally grouped into the following categories:

(a) Radioisotopes, such as ³⁵S, 14C, ¹²⁵I, ³H, and ¹³¹I. Isotopic labeling is described, for example, in Current Protocols in Immunology, Volumes 1 and 2, Coligen et al., Ed. Wiley-Interscience, New York, N.Y., Pubs. (1991), and radioactivity can be measured using any suitable detection technique, including scintillation counting.

(b) Fluorescent labels, such as rare earth chelates (europium chelates) or fluorescein and its derivatives, rhodamine and its derivatives, dansyl, Lissamine, phycoerythrin and Texas Red. A fluorescent label can be conjugated to a detection reagent, e.g., an anti-NMNAT2 antibody, using any suitable technique, and fluorescence can be quantified using, for example, a fluorimeter.

(c) Various enzyme-substrate labels are available and U.S. Pat. No. 4,275,149 provides a review of some of these. The enzyme generally catalyzes a chemical alteration of the chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Techniques for quantifying a change in fluorescence are described above. The chemiluminescent substrate may, for example, become electronically excited by a chemical reaction and may then emit light that can be measured (using a chemiluminometer, for example) or donates energy to a fluorescent acceptor. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, beta-galactosidase, glucoamylase, lysozyme, saccharide oxidases (e.g., glucose oxidase, galactose oxidase, and glucose-6-phosphate dehydrogenase), heterocyclicoxidases (such as uricase and xanthine oxidase), lactoperoxidase, microperoxidase, and the like. Techniques for conjugating enzymes to detection reagents such as anti-NMNAT2 antibodies are well known in the art. See, e.g., U.S. Pat. Nos. 4,275,149 and 4,318,980.

Sometimes, a detectable label may be indirectly conjugated with a detection reagent such as an antibody. The skilled artisan will be aware of various techniques for achieving this. For example, an antibody can be conjugated with biotin and any of the three broad categories of labels mentioned above can be conjugated with avidin, or vice versa. Biotin binds selectively to avidin and thus, the label can be conjugated with the antibody in this indirect manner. Alternatively, to achieve indirect conjugation of a label with a detection reagent (e.g., an antibody), the detection reagent is conjugated with a small hapten (e.g., digoxin) and one of the different types of labels mentioned above is conjugated with an anti-hapten antibody (e.g., anti-digoxin antibody).

The detection reagents of the present invention may be employed in any known assay method, such as competitive binding assays, direct and indirect sandwich assays, and immunoprecipitation assays. Competitive binding assays rely on the ability of a labeled standard to compete with the test sample analyte for binding with a limited amount of antibody. The amount of NMNAT2, for example, in the test sample is inversely proportional to the amount of standard that becomes bound to the anti-NMNAT2 antibodies. To facilitate determining the amount of standard that becomes bound, the antibodies generally are insoluble before or after the competition, so that the standard and analyte that are bound to the antibodies may conveniently be separated from the standard and analyte that remain unbound.

Sandwich assays involve the use of two antibodies, each capable of binding to a different immunogenic portion, or epitope, of the protein to be detected, e.g., hNMNAT. In a sandwich assay, the test sample analyte (e.g., hNMNAT2) is bound by a first anti-MNAT2 antibody (or antigen-binding antibody fragment) that is immobilized on a solid support, and thereafter a second antibody (or antigen-binding antibody fragment) binds to the analyte, thus forming an insoluble three-part complex. See, e.g., U.S. Pat. No. 4,376,110. The second antibody may itself be labeled with a detectable label (direct sandwich assays) or may be measured using an anti-immunoglobulin antibody that is labeled with a detectable label (indirect sandwich assay). For example, one type of sandwich assay is an ELISA assay, in which case the detectable moiety is an enzyme.

For immunohistochemistry, the blood or tissue sample may be fresh or frozen or may be embedded in paraffin and fixed with a preservative such as formalin, for example.

The detection reagents of the invention may also be used for in vivo diagnostic assays. Generally, the antibody is labeled with a radionuclide (such as ¹¹¹In, ⁹⁹Tc, ¹⁴C, ¹³¹I, ¹²⁵I, ³H, ³²P, or ³⁵S) so that the bound target molecule can be localized using immunoscintillography.

As a matter of convenience, a detection reagent of the present invention (e.g., an anti-NMNAT2 antibody or NMNAT2-binding antibody fragment) can be provided in a kit, for example, a packaged combination of reagents in predetermined amounts with instructions for performing a diagnostic assay. Where the detection reagent is labeled with an enzyme, the kit will include substrates and cofactors required by the enzyme (e.g., a substrate precursor which provides the detectable chromophore or fluorophore). In addition, other additives may be included such as stabilizers, buffers (e.g., a block buffer or lysis buffer) and the like. The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration

In the context of therapeutic applications of the invention, proteins or polypeptides exhibiting NMNAT activity, particularly hNMNAT2 enzymatic activity, can be delivered to diseased cells (e.g., cancer cells) expressing lower than normal levels of such activity in order to sensitize such cells to tiazofurin administration. NMNAT activity, particularly hNMNAT2 enzymatic activity, can be delivered in any suitable way. Gene therapy-based approaches and administration of recombinantly produced, enzymatically active NMNAT (e.g., rhNMNAT) are particularly preferred. For gene therapy, a gene delivery vehicle carrying an expression cassette on a nucleic acid vector that encodes the desired NMNAT activity is manufactured using suitable techniques. Examples of suitable gene delivery vehicles include viral vectors (e.g., retroviral viral vectors, including lentiviral vectors, adenoviral (AV) vectors, adeno-associated viral (AAV) vectors), so-called “naked DNA” vectors, and physical or chemical methods such as liposome-mediated nucleic acid delivery.

Depending upon the application, expression of the desired NMNAT structural gene may be transient or stable, depending upon the gene delivery vehicle used to deliver the expression cassette carried therein.

Any gene coding for a desired NMNAT activity useful in practicing the invention, preferably hNMNAT2 (see, e.g., Yalowitz, et al. [2], and the examples below for a representative description of isolating hNMNAT2), can be included in the expression cassette as the structural gene to be expressed following delivery to cells.

With regard to chemical methods for gene delivery, liposomes are preferred. Liposomes containing an expression construct coding for a desired, tiazofurin-sensitizing NMNAT activity can be prepared by methods known in the art. Liposomes typically are spherical, self-enclosed vesicles composed of amphipathic lipids, and have been widely studied and are employed as vectors for in vivo administration of therapeutic agents. In preferred embodiments, long-circulating liposomes are used that include a surface coat of flexible water soluble polymer chains, which act to prevent interaction between the liposome and the plasma components which play a role in liposome uptake o cell targeting. Alternatively, hyaluronan has been used as a surface coating to maintain long circulation. In some embodiments, liposomes encapsulate the desired expression cassette or even a viral particle. In some embodiments, the nucleic acid component of the gene delivery vehicle can be condensed with a cationic polymer, e.g., PEI, polyamine spermidine, and/or spermine, or a cationic peptide, e.g., protamine and poly-lysine, and encapsulated in the lipid particle. If desired, liposomes can comprise multiple layers assembled in a step-wise fashion.

Lipid materials are well known and routinely used in the art to produce liposomes. Lipids may include relatively rigid varieties, such as sphingomyelin, or fluid types, such as phospholipids having unsaturated acyl chains. “Phospholipid” refers to any one phospholipid or combination of phospholipids capable of forming liposomes. Phosphatidylcholines (PC), including those obtained from egg, soy beans or other plant sources or those that are partially or wholly synthetic, or of variable lipid chain length and unsaturation are suitable for use in the present invention. Synthetic, semisynthetic and natural product phosphatidylcholines including, but not limited to, distearoylphosphatidylcholine (DSPC), hydrogenated soy phosphatidylcholine (HSPC), soy phosphatidylcholine (soy PC), egg phosphatidylcholine (egg PC), hydrogenated egg phosphatidylcholine (HEPC), dipalmitoylphosphatidylcholine (DPPC) and dimyristoylphosphatidylcholine (DMPC) are suitable phosphatidylcholines for use in this invention. All of these phospholipids are commercially available. Further, phosphatidylglycerols (PG) and phosphatic acid (PA) are also suitable phospholipids for use in the present invention and include, but are not limited to, dimyristoylphosphatidylglycerol (DMPG), dilaurylphosphatidylglycerol (DLPG), dipalmitoylphosphatidylglycerol (DPPG), distearoylphosphatidylglycerol (DSPG) dimyristoylphosphatidic acid (DMPA), distearoylphosphatidic acid (DSPA), dilaurylphosphatidic acid (DLPA), and dipalmitoylphosphatidic acid (DPPA). Distearoylphosphatidylglycerol (DSPG) is the preferred negatively charged lipid when used in formulations. Other suitable phospholipids include phosphatidylethanolamines, phosphatidylinositols, sphingomyelins, and phosphatidic acids containing lauric, myristic, stearoyl, and palmitic acid chains. For the purpose of stabilizing the lipid membrane, it is preferred to add an additional lipid component, such as cholesterol. Preferred lipids for producing liposomes according to the invention include phosphatidylethanolamine (PE) and phosphatidylcholine (PC) in further combination with cholesterol (CH). According to one embodiment of the invention, a combination of lipids and cholesterol for producing the liposomes of the invention comprise a PE:PC:Chol molar ratio of 3:1:1. Further, incorporation of polyethylene glycol (PEG) containing phospholipids is also contemplated by the present invention.

In addition, in order to prevent the uptake of the liposomes into the cellular endothelial systems and enhance the uptake of the liposomes into the tissue of interest, the outer surface of the liposomes may be modified with a long-circulating agent. The modification of the liposomes with a hydrophilic polymer as the long-circulating agent is known to enable to prolong the half-life of the liposomes in the blood.

Liposomes for use in practicing the invention can be generated by any suitable method, including a reverse phase evaporation method (see, e.g., U.S. Pat. No. 4,235,871) with a lipid composition comprising phosphatidyl choline, cholesterol and PEG-derivatized phosphatidylethanolamine (PEG-PE). Infusion procedures or detergent dilution methods, among other known in the art, can also be used to form liposomes. Preferably, liposomes are extruded through filters of defined pore size to yield liposomes with the desired diameter.

The use of an therapeutically effective amount of a gene delivery vehicle, protein, or polypeptide described herein for the treatment of cancer should be preferably include but is not limited to compositions of the invention in lactated Ringer's solution. Preferably, the composition is sterile, and may be either a liquid or solid (e.g., lyophilized) composition. As will be appreciated, solid compositions are typically reconstituted in a suitable diluent prior to administration to a subject. Lactated Ringer's solution is a solution that is isotonic with blood and intended for intravenous administration. Antioxidants, buffers, and solutes that render the compositions substantially isotonic with the blood of an intended recipient are also preferably included in compositions.

In some embodiments, the compositions comprise gene delivery vector as described herein. In other embodiments, the compositions comprise a biologically NMNAT protein (e.g., rhNMNAT) or polypeptide produced by recombinant techniques, which ca include, for example, expression in mammalian (e.g., CHO, COS), insect, yeast, cell-free, or bacterial host cell expression systems. The therapeutic compositions of the invention may also include water, polyols, glycerine and vegetable oils, and nutrients for cells, for example. Compositions adapted for parenteral administration can be presented in unit-dose or multi-dose containers, in a pharmaceutically acceptable dosage form. Such dosage forms, along with methods for their preparation, are known in the pharmaceutical and cosmetic art. HARRY'S COSMETIC LOGY (Chemical Publishing, 7th ed. 1982); REMINGTON'S PHARMACEUTICAL SCIENCES (Mack Publishing Co., 18th ed. 1990).

In some embodiments, dosage forms include pharmaceutically acceptable carriers that are inherently nontoxic and nontherapeutic. Examples of such carriers include ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, zinc salts, colloidal silica, magnesium trisilicate, polyvinyl pyrrolidone, cellulose-based substances, and polyethylene glycol.

In these and other embodiments, other ingredients can also be added, including antioxidants, e.g., ascorbic acid; low molecular weight (less than about ten amino acid residues) polypeptides, e.g., polyarginine or tripeptides; proteins, such as serum albumin, gelatin, or immunoglobulins; hydrophilic polymers such as polyvinylpyrrolidone; amino acids, such as glycine, glutamic acid, aspartic acid, or arginine; monosaccharides, disaccharides, and other carbohydrates including cellulose or its derivatives, glucose, mannose, or dextrins; chelating agents such as EDTA; and sugar alcohols such as mannitol or sorbitol.

The instant compositions can be administered by any suitable route, and method, for example, parenterally, intravenously, intra-arterially, intracranially, intracerebrospinally, intratumorally, peritoneally, by injection, by catheter, by implantation with or without a matrix or gel material, or by a sustained release delivery device. In preferred embodiments, the therapeutic compositions described herein are administered directly by injection.

Pharmaceutical compositions can also be provided in the form of a combined preparation, for example, as an admixture of one or more distinct chemical species, alone in conjunction with and one or more other therapeutic agent species that are not encode or provide a desired NMNAT activity, for example, one or more chemotherapeutic agents.

A “combined preparation” includes a “kit of parts” in the sense that the combination partners can be dosed independently or by use of different fixed combinations with distinguished amounts of the two or more agent species, i.e. simultaneously, separately, or sequentially. The parts of the kit can then, for example, be administered simultaneously or chronologically staggered, that is, at different time points, with equal or different time intervals, and/or in the same or different numbers of dosings for any part of the combination.

In some embodiments, a combined preparation is administered, wherein two or more separate compositions are administered to a subject, wherein the first composition comprises a therapeutically effective amount of a desired NMNAT activity (e.g., an expression cassette that encodes hNMNAT2 or isolated or purified rhNMNAT) and the second composition comprises a therapeutically effective amount of a chemotherapeutic drug or pro-drug, for example, tiazofurin. In other embodiments, one or more additional compositions each comprising a different active ingredient can be administered.

Pharmaceutical compositions of the invention are provided for combined, simultaneous, separate, sequential, or sustained administration. In some embodiments, a composition comprising therapeutically effective amount of a desired NMNAT activity (e.g., an expression cassette that encodes hNMNAT2 or isolated or purified rhNMNAT) is administered at or about the same time as one or more chemotherapeutic agents, e.g., tiazofurin. In one embodiment, a composition comprising a desired NMNAT activity is administered within at least about thirty, sixty, ninety, or one hundred twenty minutes, or about 6, 12, 24, 48, or 168 hours of one or more of tiazofurin.

For the prevention or treatment of disease, the appropriate dosage of an active pharmaceutical ingredient (API) will depend on the type of disease to be treated, as described in this specification, the severity and course of the disease, whether the API is administered for preventive or therapeutic purposes, previous therapy, the patient's clinical history and response to the previous treatment, and the discretion of the attending physician. The API is suitably administered to the patient at one time or over a series of treatments.

Depending on the type and severity of the disease, about 0.1 microgram per kilogram (ug/kg) to about 50 mg/kg (e.g., 0.1-20 mg/kg) of API is an initial candidate dosage for administration to the patient, whether, for example, by one or more separate administrations, or by continuous infusion. A typical daily or weekly dosage might range from about 1 μg/kg to about 20 mg/kg or more, depending on the factors mentioned above and/or otherwise known in the art. For repeated administrations over several days or longer, depending on the condition, the treatment is repeated until a desired suppression of disease symptoms occurs. However, other dosage regimens may be useful. The progress of this therapy is easily monitored by conventional techniques and assays, including, for example, radiographic imaging. Diagnostic methods, including companion diagnostic methods, used to determine NMNAT activity or levels in bodily fluids or tissues may be used in order to optimize patient exposure to the therapeutic agents of the invention.

In another aspect of the invention, an article of manufacture, or kit, containing materials useful for the treatment of the disorders described above is provided. The article of manufacture comprises a container and a label. Suitable containers include, for example, bottles, vials, syringes, and test tubes. The containers may be formed from a variety of materials such as glass or plastic. The container holds a composition which is effective for treating the condition and may have a sterile access port (for example the container may be an intravenous solution bag or a vial having a stopper pierceable by a hypodermic injection needle). The label on, or associated with, the container indicates that the composition is used for treating the condition of choice. The article of manufacture may further comprise a container comprising a pharmaceutically-acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

2. Folate Receptors and Targeted Drug Delivery

Folate receptors (FR) are glycosylphosphatidylinositol (GPI)-anchored glycoproteins that are overexpressed in 30-50% of colorectal carcinomas [17] and at varying frequencies in other cancers [18]. Folate is a high affinity ligand for the FR and hence folate has become one of the most investigated targeting ligands for tumor-specific delivery of therapeutic agents to cancer cells via FR [19-21]. FR targeting is used here to transport tiazofurin into FR over-expressing cancer cells that can metabolize tiazofurin intracellularly to TAD to induce cancer cell killing.

Nanoparticles such as liposomes are bio-degradable, biologically inert, enclosed self-forming bi-layer lipids and when hydrated become efficient carriers of drugs for the treatment of cancer. Nanoparticle-based drug delivery systems allow for targeted delivery and an enhanced therapeutic index for chemotherapeutic agents useful in killing cancer cells but which have reduce toxicity to normal cells because of the ease at which their surface can be modified compared to microemulsions [22].

Pro-drugs are pharmacologically inactive derivatives of active drugs. Gene-directed enzyme pro-drug therapy, also known as “suicide gene therapy”, in which an exogenously administered gene (enzyme) is delivered to tumor cells. The gene is then expressed intratumorally, which converts the pro-drug to its active form in those cells that express the “suicide” gene, resulting in enhanced cell killing. A selective gene directed enzyme pro-drug therapy is an attractive alternate to conventional drug therapy, which has drawbacks that include low achievable drug concentrations in tumor cells and systemic toxicity to the host. Recently, successful in vitro and in vivo cancer cell-kill with gene-directed enzyme pro-drug therapy systems, such as Herpes simplex virus thymidine kinase with ganciclovir [23] and E. coli cytosine deaminase with 5-fluorocytosine [24], have been demonstrated. For this approach to be successful in clinic, the enzyme should be of human origin to express the protein of interest that is absent or expressed only at low concentrations in normal tissues and the pro-drug should be a good substrate for the expressed enzyme. The effective drug should be diffusible and actively taken up by adjacent cancer cells to exhibit bystander effect.

The Examples below describe experiments aimed at examining the relationship of hNMNAT2 expression with cell-kill to tiazofurin in colorectal cancer cell lines. In addition, the effect of transfecting hNMNAT2 into colorectal cancer cells was investigated. Overexpression of hNMNAT2 was confirmed to be directed to cancer cell cytoplasm. The specificity of hNMNAT2 and its influence on cell-kill by tiazofurin was established using RNA interference studies. Since FR are expressed on the colorectal cancer cell surface, its utility in targeted transport of tiazofurin through cancer cell membrane to influence cell-kill was also examined as a way of targeting cancer cells that express FR, including colorectal cancer cells. Other cancer cell types that overexpress FR include those of many human cancers, including breast, ovarian, lung, and renal cancers, as well as pediatric ependymal brain tumors, mesothelioma, myeloid leukemia, and head and neck carcinomas. In short, any cancer cell type now known or discovered to overexpress FR can be targeted in accordance with the invention.

Adapting this approach to target other cancer cell types that overexpress a cell surface antigen can be readily accomplished by changing the targeting moiety of the nanoparticles. For example, nanoparticles that include human carcinoembryonic antigen (CEA) can be used to target colorectal carcinomas, gastric carcinomas, pancreatic carcinomas, lung carcinomas, breast carcinomas, and thyroid carcinomas, as CEA has been found to be overexpressed in such cancers as compared to CEA levels in healthy individuals (e.g., CEA levels above 2.5 ng/mL). Similarly, alphafetoprotein (AFP) can be used to target germ cell tumors and hepatocellular carcinoma, CA-125 to target ovarian cancer, MUC-1 and/or epithelial tumor antigen (ETA) to target breast cancer, and tyrosinase and/or MAGE to target malignant melanoma.

### EXAMPLES

The invention will be further described by reference to the following detailed examples. These Examples are in no way to be considered to limit the scope of the invention in any manner.

Unless otherwise stated, the present invention can be performed in view of this specification using standard procedures, as described, for example in Maniatis, et al., Molecular Cloning: A Laboratory Manual, Cold Spring Harbor Laboratory Press, Cold Spring Harbor, N.Y., USA (1982); Sambrook, et al., Molecular Cloning: A Laboratory Manual (2 ed.), Cold Spring Harbor Laboratory Press, Cold Spring Harbor, N.Y., USA (1989); Davis, et al., Basic Methods in Molecular Biology, Elsevier Science Publishing, Inc., New York, USA (1986); Methods in Enzymology: Guide to Molecular Cloning Techniques Vol. 152, S. L. Berger and A. R. Kimmerl Eds., Academic Press Inc., San Diego, USA (1987); Current Protocols in Molecular Biology (CPMB) (Fred M. Ausubel, et al. ed., John Wiley and Sons, Inc.); Current Protocols in Protein Science (CPPS) (John E. Coligan, et. al., ed., John Wiley and Sons, Inc.); and Current Protocols in Immunology (CPI) (John E. Coligan, et. al., ed. John Wiley and Sons, Inc.), which are all incorporated by reference herein in their entireties.

### Materials and Methods

### Materials

Unless specified, all chemicals and reagents of the highest purity were obtained from the Sigma-Aldrich, St. Louis, Mo.

### Cell Culture

Human colorectal cancer cell lines HCT15, HCT116, HT29, HCC2998, and Caco2 were obtained from the National Cancer Institute, Bethesda, Md., USA, maintained in 5% CO₂ at 3TC in minimum essential media containing 2.3 μM folic acid (MEM, Life Technologies, Inc., Grand Island, N.Y.) supplemented with 1% penicillin, streptomycin, amphotericin B (Invitrogen, Carlsbad, Calif.), and 10% fetal bovine serum (FBS) for all cell lines except Caco2, which required 20% FBS for optimal growth. Caco2 and HT29 cells were adapted to grow in a low-folate (LF) condition by step-wise reduction of the folate concentration in the medium and finally were stably adapted to grow in folate-free MEM supplemented with 20% and 10% FBS, respectively [25]. The complete LF medium with 20% FBS for Caco2 cells contained 18 nM 5-methyltetrahydrofolate and the LF medium with 10% FBS for HT29 cells contained 9 nM 5-methyltetrahydrofolate [25]. The cells were cultured in low 5-methyltetrahydrofolate conditioned medium for at least 20 generations before they were used in the experiments to promote full expression of folate receptors, and logarithmically growing cells were utilized for all studies.

### Establishment of Stable Transfected Cell Lines

The hNMNAT2 gene was amplified using primers 5′-GGGGTACCARGACCGAGACCACCAAGAC-3′ (SEQ ID NO: 1) and 5′-GCTCTAGAATCGATGCTAGCCGGAGGCATTG-3′ (SEQ ID NO: 2) [2]. The PCR product was digested by KpnI and XbaI and cloned into the vector pTracerEFV5/HisA (Invitrogen, Carlsbad, Calif.). The correct plasmid pTracer-hNMNAT2 constructs were confirmed by restriction enzyme analysis and sequencing.

Colorectal cancer cells were transfected with pTracer hNMNAT2 and the control plasmid pTracerEFV5/HisA using the lipofectamine reagent (Invitrogen, Carlsbad, Calif.). Zeocin resistant green fluorescence protein (GFP) expressing clones were further analyzed by Fluorescence-activated cell sorting (Becton Dickinson Immunocytometry Systems, San Jose, Calif.).

### Cytoplasmic, Mitochondrial and Nuclear Protein Extraction

Nuclear and cytoplasmic fractions of colorectal cancer cells were separated and purified using Pierce protein separation kit as per manufacturer's instructions. For mitochondrial protein extraction, colorectal cancer cells were extracted with Tris-sucrose (pH 7.4) buffer as per published methodology [26]. Mitochondrial specific marker, MTC 02 antibody (Abcam, Cambridge, Mass.) was used to show that overexpression of hNMNAT2 was confined to cytoplasm.

Protein concentration from the above samples was determined by BCA protein assay kit (Pierce BCA protein assay kit, Thermo Fisher Scientific, Inc., Rockford, Ill.).

Generation of hNMNAT2 Silenced Caco2 Cell Lines to Demonstrate Consequences of hNMNAT2 Overexpression

Colorectal cancer Caco2 cells overexpressing hNMNAT2 were transfected with control shRNA plasmid and NMNAT2 small hairpin shRNA plasmid (sc-62693-SH: NMNAT2 shRNA Plasmid (h) is a pool of 3 different shRNA plasmids with a hairpin sequence: GATCCGTAGAAAGTGAGACTCAATTTCAAGAGAATTGAGTCTCACTTTCTACTTTTT (SEQ ID NO: 3) and sc-62693-SHA: 5′-GUAGAAAGUGAGACUCAAUtt-3′ (SEQ ID NO: 4) and 5′-AUUGAGUCUCACUUUCUACtt-3′ (SEQ ID NO: 5); sc-62693-SHB: 5′-CCUUAGGAAUAGCAUUGUAtt-3′ (SEQ ID NO: 6) and 5′-UACAAUGCUAUUCCUAAGGtt-3′ (SEQ ID NO: 7); sc-62693-SHC: 5′-GCUCUUUCCCUCAACCUUAtt-3′ (SEQ ID NO: 8) and 5′-UAAGGUUGAGGGAAAGAGCtt-3′ (SEQ ID NO: 9). Transfection was conducted for 8 h as per manufacturer's instructions (Santa Cruz Biotechnology, Inc., Santa Cruz, Calif.). Puromycin resistant clones were selected and knockdown of hNMNAT2 was confirmed by Western blot analyses.

Silencing of hNMNAT1 and hNMNAT3 Expression in Wild-Type and hNMNAT2 Overexpressing Human Colorectal Cancer Cell Lines

Initial studies were carried out to establish optimal conditions (for cell density, transfection reagent and siRNA concentrations) for transfection using siRNA [hNMNAT1 (s34981, Invitrogen, Carlbad, Calif.) or hNMNAT3 (s51395, Invitrogen, Carlbad, Calif.)] in colorectal cancer cell lines Caco2 and HT29. Briefly, 0.2×10⁶ cells were plated in a 6-well plate without antibiotics. After 24 h media was aspirated, washed with PBS and Opti-MEM media was added. For each transfection sample using Lipofectamine™2000 (Invitrogen), an aliquot of siRNA (NMNAT1 or NMNAT3) (10 pmol) of was taken in OPti-MEM (500 μl). Lipofectamine™2000 (10 μl) was diluted in Opti-MEM (500 μl) mixed gently and incubated for 5 min at room temperature. After 5 min the diluted oligomer was combined with the diluted Lipofectamine™2000, mixed gently and further incubated for another 20 min at room temperature before adding the complex to each well. This was incubated at 37° C. for 24 to 96 h to assay for silencing of respective genes.

Real-Time RT-PCR for Assessing hNMNAT3 mRNA Expression

Total RNA was purified from the transfected cells using GenElute™ mRNA miniprep kit from Sigma according to the manufacturers instructions. Single stranded cDNA was synthesized from total RNA using the SuperScript III system (Agilent technologies, Santa Clara, Calif.). Real time RT-PCR for each target was performed with SYBR Green (Agilent technologies, Santa Clara, Calif.) using the Agilent Mx3005P QPCR system. Primers sets for NMNAT3 were (5′-AACAACGCATTGCCATAGCTCGTG-3′; SEQ ID NO: 10) and (5′-AGTCTGCATTCTGGATGGTGGACA-3′; SEQ ID NO: 11); and for GAPDH were: (5′-AGAACATCATCCCTGCATCC-3′; SEQ ID NO: 12) and (5-AGTTGCTGTTGAAGTCGC-3′; SEQ ID NO: 13). The thermal cycling conditions consisted of pre-heating (10 minutes at 94° C.) and 40 cycles of denaturation (10 seconds at 94° C.), annealing (20 seconds at 60° C.) and elongation (20 seconds at 72° C.). Each mRNA level was normalized with the internal control GAPDH mRNA level.

### Analysis of NMNAT Activity

NMNAT activity in the cytoplasmic samples was analyzed by using a discontinuous assay system using alcohol dehydrogenase, and NADH formed was monitored at 340 nm as described [2]. Enzyme rate calculations were based on NADH extinction coefficient of 6220 M⁻¹ cm⁻¹. One unit of enzyme activity was defined as the capacity to form 1 pmol of NADH per minute.

### Determination of IMPDH Activity and NAD⁺ Concentration

Colorectal cancer cells were extracted and IMPDH activity was assayed as described and activity is expressed as nmol/min/mg protein [8]. NAD concentration in Caco-2 cells was determined as cited [2].

### Western-Blot Analyses

Cytoplasmic cell extracts were prepared and 30 μg of the cell extract was separated on a 12% polyacrylamide gel and transferred to nitrocellulose membrane. Blots were blocked, incubated with primary anti-hNMNAT-2 antibody [2], anti-IMPDH-2 monoclonal antibody (Antibody Solutions, Palo Alto, Calif.) or GAPDH and then conjugated with secondary anti-mouse or anti-rabbit horseradish peroxidase antibody, and detected by enhanced chemiluminescent western blotting detection system.

### Determination of GTP and TAD Concentrations in Colorectal Cancer Cells

For the determination of GTP and TAD, colorectal cancer cells were treated with concentration of tiazofurin (specified in legends to figures) or with saline for 4 h and cell extracts were prepared as detailed earlier [27]. An aliquot of neutralized extract was injected into Phenomenex Gemini 250×4.6 mm, 5μ C18 110A column (Torrance, Calif.) maintained at 19° C., preequilibrated with buffer A (0.1 M potassium phosphate, pH 6.0) and then subjected to a stepwise gradient of buffer A with 20% acetonitrile, pH 6.0 over 30 min and analyzed on Shimadzu High Performance Liquid Chromatography (Canby, Oreg.). Under these conditions GTP eluted at 10 min separated from other nucleotides.

### Nanoparticle Preparation

Folate-tethered and non-targeted nanoparticles containing tiazofurin or calcein of the following composition were used in the study: folate-tethered nanoparticles composed of distearoylphosphatidylcholine (DSPC), cholesterol, DSPE-PEG-folate (56:40:0.1 v/v); and non-targeted nanoparticles composed of DSPC/cholesterol/PEG-DSPE (56:40:0.1 v/v). All lipids were obtained from Avanti Polar Lipids, AL. Briefly, 100 mg lipid mixture was dissolved in 3 ml chloroform and dried to a thin film in a round-bottom flask on a rotary evaporator under reduced pressure for about 1 h. The dried lipid mixture was then rehydrated with 1 ml tiazofurin (1.0 M in PBS, pH 7.4) or calcein (1 mM calcein in PBS, pH 7.4) by constantly rotating the round bottom flask at 60° C. with vortexing. The resulting suspension of multilamellar vesicles was then subjected to 10 cycles of freezing and thawing, sonicated for 10 min using an ultrasonic cleaner (EMC model 250, Hickory, N.C.) and extruded 10 times through 400, 200, and 100 nm pore size polycarbonate membrane using a stainless steel extruder (Northern Lipid, Vancouver, Canada) circulated with 60° C. water. The resulting nanoparticles encapsulating tiazofurin or calcein were then separated from the free-drug on a Sepharose CL-4B column (10×1.5 cm) pre-equilibrated with PBS. The opaque nanoparticle fractions of 2 ml were eluted in the void volume and concentration of tiazofurin were determined with 50 μl aliquots of nanoparticle preparation in Eppendorf test tubes. The tubes were centrifuged at 18,000 g for 5 min to pellet nanoparticles. An aliquot of the supernate was used to measure free tiazofurin. To the pellet, 0.5 ml of 1% aqueous sodium dodecyl sulfate solution was added to solubilize nanoparticles. An aliquot was made up to 1 ml with water and transferred to quartz cuvette and absorbance read at 238 nm in a spectrophotometer (Molecular Devices, Specra Max 250, Sunnyvale, Calif.). For measuring calcein concentrations in nanoparticles, calcein fluorescence was quantitated by excitation at 516 nm and measuring at 496 nm using a fluorescence spectrophotometer (FluoroMax-2, ISA Instruments, Edison, N.J.) [20]. The size distribution of the various nanoparticle preparations was determined by light scattering, and the median size of all nanoparticle preparations was 127±36 nm in diameter. All nanoparticle samples were stored at 4° C. and used within 2 weeks of preparation. During this period, no significant leakage of calcein or tiazofurin was detected by gel filtration, and no change of binding capacity was noted.

### Fluorescence-Activated Cell Sorting Analysis for the Uptake of Folate-Tethered and Non-Targeted Calcein Nanoparticles

To analyze calcein uptake in nanoparticles, 0.2×10⁶ cells maintained in normal or low-folate medium were cultured and folate-targeted and non-targeted nanoparticles containing calcein (1-100 μM calcein) in 0.5 ml folic acid-free medium were added. To block the entry of calcein, cells were incubated with 1 mM free folic acid (pH adjusted to 7.0) for 20 min at 37° C. prior to the addition of nanoparticles containing calcein and cells were then incubated for 2 h at 37° C. on a shaking platform. The medium from the plates was aspirated, washed and immediately analyzed on a FACScan (Becton, Dickinson Immunocytometry Systems, San Jose, Calif.).

### Cell-Kill Assay

Cytotoxicity towards tiazofurin exhibited by hNMNAT-2 stable transfectants and wild-type cell lines was assayed by using a dye-based cell proliferation assay kit (Promega, Madison, Wis., USA). Colorectal cancer cells (1500 cells/0.1 ml) were dispensed into 96-well tissue culture plates and 24 h later treated with serial dilutions of free tiazofurin or nanoparticles containing tiazofurin (for nanoparticle treatment, cells were incubated for 2 h with gentle shaking, aspirated and fresh media was added) and further incubated for 72 h at 3TC. After incubation, 20 μl of 3-(4,5-dimethylthiazol-2-yl)-5-(3-carboxymethoxyphenyl)-2-(4-sulfophenyl)-2H-tetrazolium reagent was added to each well, mixed, plates were further incubated for 2 h and color developed was read at 490 nm using a microplate spectrophotometer (Spectra MAX250, Molecular Devices, Sunnnyvale, Calif.) and cell-kill data were analyzed by Graphpad Prism 4 software (GraphPad Software, San Diego, Calif.). EC50 (Effective Concentration₅₀) of tiazofurin is defined as the concentration of the drug required to reduce the cell proliferation by 50%.

### Statistical Analysis

All experiments were conducted at least three times in triplicates. Differences between means were subjected to statistical evaluation by using Student's t test, P<0.05 was considered significant and is indicated by an asterisk.

### Results

Relationship of NMNAT Expression with Tiazofurin Cell-Kill in Colorectal Cancer Cells

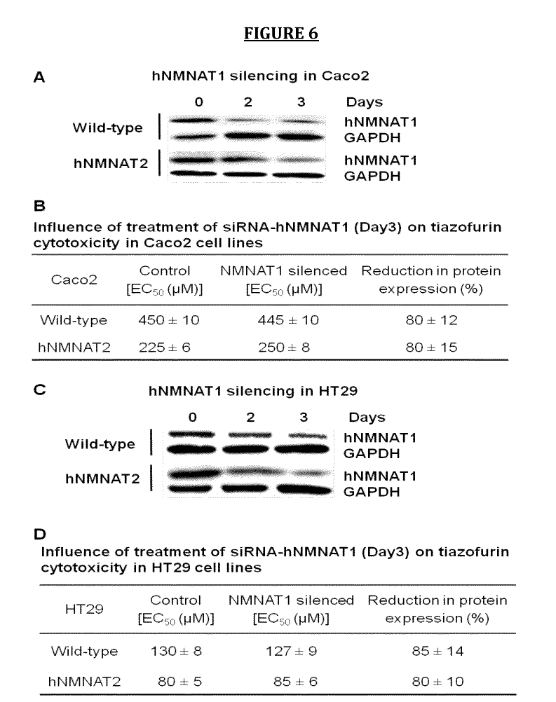

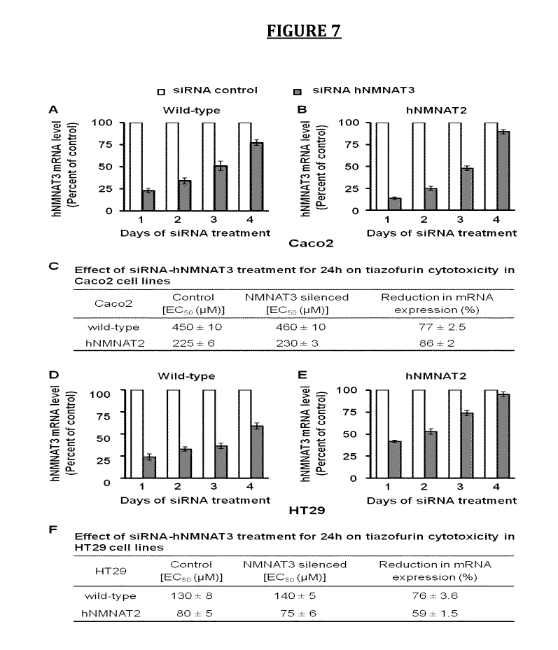

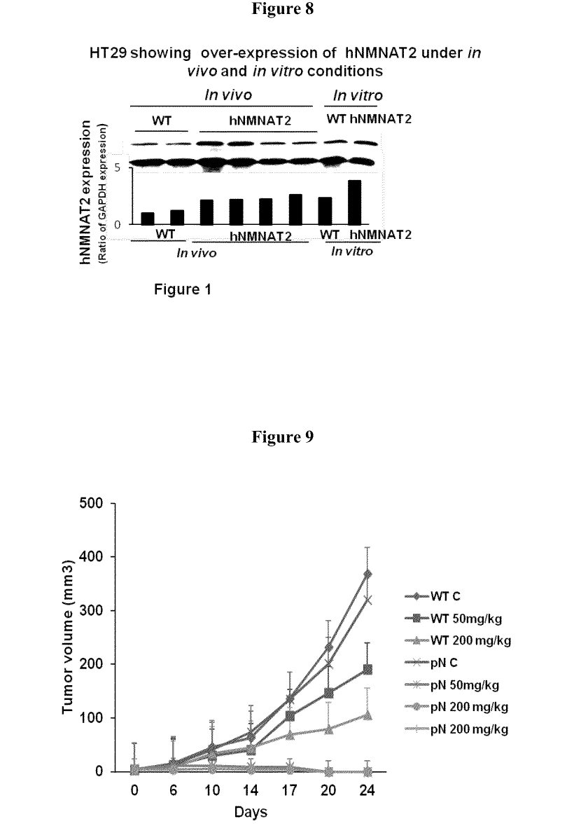

The colorectal cancer HCT15 cell line displayed the highest expression of hNMNAT2 in relation to GAPDH expression, whereas the Caco2 cell line showed the least (FIG. 2 A). Tiazofurin exhibited EC50 values of 63.9, 72.6, 124.7, 274.3, and 450.0 μM in HCT15, HCT116, HT29, HCC2998, and Caco2 cell lines, respectively (FIG. 2 B). HCT15 cells, which exhibited highest cell-kill with lower tiazofurin concentration, also showed the highest level hNMNAT2 expression, demonstrating a good correlation between hNMNAT2 expression and tiazofurin cell-kill. Moreover, the Caco2 and HT29 cell lines, which revealed the least and moderate tiazofurin cell-kill, exhibited very low and moderate hNMNAT2 expression, respectively. Thus, when the ratio of hNMNAT2 expression to tiazofurin cell-kill was subjected to regression analysis there was a significant inverse correlation between hNMNAT2 expression and tiazofurin cell-kill in these colorectal cancer cell lines with an R² value of 0.86 (FIG. 2 C). Thus, it was of interest to examine the consequences of overexpression of hNMNAT2 in colorectal cancer cell lines.

Influence of hNMNAT2 Transfection on hNMNAT2 Expression in Colorectal Cancer Cell Lines

The hNMNAT2 gene overexpression was confirmed by GFP expression by FACS analyses of Caco2 and HT29 transfected cell lines (FIGS. 2 D and 2 F). The FACS positive cells were further subjected to Western blot analyses to establish over-expression of hNMNAT2. There was a 6-fold and 3-fold increase in hNMNAT2 expression in Caco2 and HT29 cell lines, respectively when compared to their wild-type cell lines (FIGS. 2 E and 2 G).

Effect of Overexpression of hNMNAT2 in Caco2 Cells on NAD Levels and IMPDH Expression

Both biosynthetic and salvage pathways for NAD⁺ synthesis depend on NMNAT activity and perturbations in NAD⁺ levels can affect cellular metabolism and survival. Hence, NAD⁺ pools in hNMNAT2 overexpressing cell line were examined. The results (FIG. 3A) demonstrate that there was no significant change in NAD levels between wild-type and hNMNAT2 transfected cell lines. Since NAD⁺ is one of the substrates for IMPDH activity, and that IMPDH2 is the target for tiazofurin action, it was determined if IMPDH2 activity was altered in hNMNAT2 transfected cells. The results demonstrated that there was no significant difference in the expression levels of IMPDH between wild-type and hNMNAT2 transfected cell lines (FIG. 3B).

Relationship of IMPDH2 Inhibition with Cell-Kill by Tiazofurin

To delineate effect of tiazofurin treatment on IMPDH activity, wild-type and hNMNAT2 transfected Caco2 cell lines were treated with sub-lethal concentrations of tiazofurin for 4 h and IMPDH activity was determined (FIG. 3C). Since the concentrations of tiazofurin selected in this study were sub lethal to the wild-type Caco2 cell line, there was no reduction in IMPDH activity in wild-type cell line, while hNMNAT2 transfected cell line exhibited a significant decrease in IMPDH activity with an EC50 of 250 μM. Moreover, if there is relationship of IMPDH inhibition to tiazofurin cell-kill, then tiazofurin levels for an effective cell-kill would also be in the range of 250 μM in hNMNAT2 transfected cell line. Thus, the effect of tiazofurin was examined on cell-kill exhibited by wild-type and hNMNAT2 transfected cell lines. As indicated (Table 1, below), the EC50 concentration of tiazofurin was reduced about 2-fold in hNMNAT-2 transfected Caco2 and HT29 cell line lines.

TABLE 1
Effect of tiazofurin on cell-kill in wild-
type and hNMNAT2 transfected cell line

| | Cytotoxicity [EC₅₀ (μM)] | | |
|---|---|---|---|
| Cell line | Wild-type | hNMNAT2 | Fold change |
| Caco2 | 450 ± 10 | 225 ± 6* | 1.8 |
| HT29 | 130 ± 8 | 80 ± 3* | 1.6 |

Experimental details are provided in the Methods section.
*Denotes statistical significance (P < 0.05)

These studies show that cytotoxic action of tiazofurin was in good agreement with IMPDH inhibitory activity in hNMNAT2 transfected cell lines.

Localization of overexpression of hNMNAT2 to cytoplasm and effect of silencing hNMNAT2 overexpression

To confirm hNMNAT2 protein overexpression was related to NMNAT enzyme activity, wild-type and hNMNAT2 overexpressing Caco2 cell lines were subjected to cytoplasmic extraction and NMNAT activity was assayed. The results showed a 2-3-fold increased activity that correlated with hNMNAT2 protein overexpression (FIG. 3D).

To examine if overexpression of hNMNAT2 in colorectal cancer cells was confined to cytoplasm, wild-type and hNMNAT2 overexpressing Caco2 cell lines were subjected to differential extraction procedures for mitochondrial, nuclear, and cytoplasmic proteins. The results demonstrated that there was a 2-3-fold higher expression of hNMNAT2 in the cytoplasm of hNMNAT2 overexpressing Caco2 cell line and no overexpression of hNMNAT2 was noted in other cellular compartments (FIG. 3E).

To further investigate if overexpression in colorectal cancer cells was due to hNMNAT2, attempts were made to silence hNMNAT2 overexpression in the Caco2 cell line by transfection with hNMNAT2-shRNA. Silencing of overexpression of hNMNAT2 was confirmed following 48 h exposure (FIG. 3F). Knockdown cells were then selected with puromycin and further examined to determine the influence of tiazofurin treatment (FIG. 3G). Wild-type Caco2 cells exhibited an EC50 of 450 μM, whereas hNMNAT2 transfected cells showed a 2-fold reduction in EC50 value (225 μM). hNMNAT-2-shRNA knockdown cells showed a partial reversal of cell-kill by tiazofurin with an EC50 value of 380 μM, indicating that sensitivity to tiazofurin was related to overexpression of hNMNAT2 in these cell lines.

Effect of Tiazofurin Treatment on GTP Concentration in Wild-Type and hNMNAT2 Overexpressing Colorectal Cancer Cell Lines

To examine the influence of tiazofurin treatment on GTP levels in Caco2 and HT29 cell lines, Caco2 cell lines were incubated with saline (control) or 150 or 300 μM tiazofurin for 4 h. There was a 29% and 45% reduction in GTP pools following treatment with 150 and 300 μM tiazofurin, respectively, in wild-type Caco-2 cells (FIG. 4A), whereas the hNMNAT2-transfected Caco2 cell line treated with similar concentrations of tiazofurin resulted in greater reduction (40% and 60%) in GTP levels (FIG. 4B). Reduction in GTP levels correlated with 2-fold higher concentration of TAD in hNMNAT2 overexpressing cell line (FIG. 4C). HT29 cell lines were incubated with saline (control) or 25 or 100 μM tiazofurin for 4 h. Tiazofurin treatment did not result in GTP reduction in wild-type cell line treated with 25 μM concentration and even when treated with 100 μM tiazofurin GTP reduction was 22% (FIG. 4D). However, tiazofurin treatment in the hNMNAT2-transfected HT29 cell line showed a sharper reduction in GTP levels: 21% and 47% at 25 and 100 μM tiazofurin concentrations, respectively (FIG. 4E), concurring with 2-fold higher levels of TAD in the hNMNAT2-overexpressing HT29 cell line (FIG. 4F). These studies demonstrate that tiazofurin treatment resulted in greater reduction in GTP levels in hNMNAT-2 transfected colorectal cancer cell lines when compared to wild-type cell lines and this effect correlated to TAD levels found following tiazofurin treatment.

### Cellular Uptake of Nanoparticles Containing Calcein Via Folate Receptors in Colorectal Cancer Cell Lines

The specificity of calcein uptake as a measure of FR expression was examined in Caco2 and HT29 cell lines cultured in low-folate and then incubated with calcein for 90 min. FACScan analysis indicated that there was 3-fold (FIG. 5A) and 2-fold (FIG. 5D) higher uptake of calcein encapsulated in folate-tethered nanoparticles by Caco2 and HT29 cell lines, respectively, when compared to calcein in non-targeted nanoparticles. The addition of folic acid prior to nanoparticle incubation decreased the mean value of calcein fluorescence uptake in folate-tethered nanoparticles (results not shown) suggesting calcein uptake was mediated via folate receptors. There was a dose-dependent increase in calcein uptake in folate-tethered nanoparticles by colorectal cancer cell lines [20].

### Cell-Kill by Tiazofurin Encapsulated in Folate-Tethered Nanoparticles

Previous studies have shown that FR is upregulated when human cervical carcinoma HeLa cells were grown in low-folate medium [20,25]. Therefore, Caco2 and HT29 cell lines were adapted to grow in low-folate medium and incubated with tiazofurin encapsulated in folate-tethered and non-targeted nanoparticles. Folate-tethered nanoparticles containing tiazofurin exhibited EC50 cell-kill value of 220 μM compared to non-targeted nanoparticles that showed EC50 cell-kill value of 415 μM in wild-type Caco2 cells (FIG. 5B). This result indicated a 2-fold reduction in tiazofurin required for inducing similar cell-kill in wild-type Caco2 cell line when the drug was encapsulated in folate-targeted compared to non-targeted nanoparticles. Wild-type Caco2 cell line exhibited a 3-fold lesser level of tiazofurin requirement to induce similar cell-kill when encapsulated in folate-tethered nanoparticles. This concentration was further reduced 2-fold in hNMNAT2 transfected cell line (bringing the total to 6-fold) by encapsulating tiazofurin in folate-tethered nanoparticles (FIG. 5C). Similar results were also observed with HT29 cell lines. The wild-type HT29 cell line required 2-fold less tiazofurin encapsulated in folate-tethered nanoparticles than tiazofurin encapsulated in non-targeted nanoparticles (FIG. 5E). The HT29 cell line transfected with hNMNAT2 required 2-fold less tiazofurin than the wild-type HT29 cell line to exhibit similar cell-kill for tiazofurin encapsulated in non-targeted nanoparticles (FIG. 5F). Thus, in a hNMNAT2-transfected HT29 cell line the concentration of tiazofurin was further decreased by 3-fold (bringing the total to 6-fold) when encapsulated in folate-tethered nanoparticles.

Influence of Silencing hNMNAT1 on Cell-Kill by Tiazofurin

To examine the specificity of overexpression of hNMNAT2 on cell-kill by tiazofurin, hNMNAT1 expression in wild-type and hNMNAT2 overexpressing human colorectal carcinoma cell lines was examined by silencing hNMNAT1 using siRNA against hNMNAT1. The results showed that siRNA-hNMNAT1 treatment for 3 days resulted in a 80% reduction in hNMNAT1 expression in wild-type and hNMNAT2 overexpressing Caco2 cell lines (FIG. 6A). The effect of this silencing of hNMNAT1 expression on tiazofurin cell-kill was then examined. The results showed that silencing of hNMNAT1 protein expression did not have any influence on cell-kill by tiazofurin in either wild-type or hNMNAT2 overexpressing Caco2 cell lines (FIG. 6B). Similarly, treatment with siRNA-hNMNAT1 for 3 days in wild-type and hNMNAT2-overexpressing HT29 cell lines resulted in 80% to 85% reduction, respectively, in hNMNAT1 expression (FIG. 6C). Consequence of silencing hNMNAT1 on tiazofurin cell-kill was next examined. The results demonstrated that silencing of hNMNAT1 had no effect on tiazofurin cell-kill in either wild-type or hNMNAT2-overexpressing HT29 cell lines (FIG. 6D), similar to that observed with Caco2 cell lines.

Consequences of Silencing hNMNAT3 on Cell-Kill by Tiazofurin

The outcome of silencing hNMNAT3 on tiazofurin cell-kill in colorectal cancer cell lines was then examined. Wild-type (FIG. 7A) and hNMNAT2 overexpressing (FIG. 7B) Caco2 cell lines were treated with siRNA-hNMNAT3 for periods up to 4 days. In this case, one day treatment resulted in 80% to 90% reduction in mRNA expression of hNMNAT3 in both cell lines. The effect of silencing hNMNAT3 on wild-type and hNMNAT2 overexpressing Caco2 cell lines was examined and the results showed no change in the dose of tiazofurin required to provide 50% cell-kill (FIG. 7C). Parallel studies were conducted to silence hNMNAT3 expression in wild-type (FIG. 7D) and hNMNAT2 overexpressing (FIG. 7E) HT29 cell lines. In these cell lines one day treatment resulted in 62% to 85% decrease in mRNA expression of hNMNAT3 and this diminution did not influence cell-kill by tiazofurin in these cell lines (FIG. 7F). In the case of hNMNAT3 expression, it was necessary to depend on mRNA expression instead of protein expression because of technical problems in isolation and analysis of small volumes of siRNA treated cell lines.

Comparison of hNMNAT2 Expression in Wild-Type and hNMNAT2-Over-Expressed HT29 Cell Line, In Vitro and In Vivo

Wild-type and hNMNAT2-over-expressed HT29 cell lines were subcutaneously transplanted into athymic mice. Both types of tumors grew at a similar doubling time and tumor-size, reaching a diameter of 0.5 cm by day 18. Tumors were removed and extracted to determine hNMNAT2 expression by Western blots analyses (FIG. 8). The results showed a 2-fold increase in hNMNAT2 expression in tumors injected into mice with hNMNAT2-over-expressing cells in vivo, which was similar to the results observed with cells grown in vitro. These studies demonstrate stable over-expression of hNMNAT2 in cells grown in vivo.

Comparison of the Effect of Tiazofurin on Wild-Type and hNMNAT2-Over-Expressed HT29 Tumors, In Vivo

In vivo studies were conducted studies to compare antitumor activity of tiazofurin in athymic mice implanted subcutaneously with wild-type and hNMNAT2-over-expressed HT29 cell lines. Groups of five female athymic mice 6-8 weeks old, weighing 16-18 grams, obtained from Harlan Sprague Dawley, Indianapolis, Ind., were injected with wild-type or hNMNAT2-over-expressed HT29 (2×10⁶ cells/mice), and tumors were allowed to establish for 7 days. Mice were then treated intraperitoneally with saline or tiazofurin once-a-day for 10 days (QD10) and tumor volume and weights were monitored twice a week. The results of these studies are presented in FIG. 9 and show that in saline-treated control groups of mice, the tumors (both wild-type and hNMNAT2-over-expressed) grew at a similar rate and had reached tumor volume of 360-400 mm³ on day-28. There was a dose-dependent decrease in tumor volume following tiazofurin treatment in mice bearing wild-type HT29 (70% decrease in tumor volume at 200 mg/kg and 50% decrease in tumor volume at 50 mg/kg). Tiazofurin treatment of mice bearing overexpressed hNMNAT2 resulted in complete disappearance of tumor at both the 200 mg/kg and 50 mg/kg doses, perhaps resulting in ‘cure’. These studies are encouraging and show that hNMNAT2 over-expression in tumor leads to higher metabolism of tiazofurin to TAD following treatment resulting in tumoricidal effect. These studies indicate that hNMNAT2-gene-targeted tiazofurin therapy for colorectal cancer is feasible. Further decreasing the dose of tiazofurin will be possible by targeting FR and/or CEA and encapsulating tiazofurin in folate-tethered nanoparticles. Further targeting would translate to additional lowering of tiazofurin induced side-effects.

### Discussion

Colorectal cancer is the second most common cause of death from cancer in men and women in the United States with 102,900 new cases in 2010. It is notable that 40-50% of patients who undergo potentially curative surgery alone eventually relapse and die of metastatic disease [28]. Standard therapy in metastatic colon cancer, which comprises a chemotherapeutic combination that includes 5-fluorouracil, leucovorin, and oxaliplatin plus anti-vascular endothelial growth factor monoclonal antibody as first line treatment results in a median survival of 10-15 months [29-31]. Since conventional therapy is relatively non-specific and cytotoxicity occurs in both tumor and normal cells, there is need for selective targeting of colorectal cancer with sparing of normal cells.

Three human isoforms of NMNAT with tissue specificity are known but isoform specific functions are still emerging [32,33]. Studies on expression patterns of human NMNATs in red blood cells have suggested absence of hNMNAT-2 and its implications are yet to be determined [34]. Recent gene array studies show reduced NMNAT2 levels in brain specimens from patients with Alzheimer's disease [35]. Of the three homologs of hNMNAT, hNMNAT2 was found to be the most labile with a half-life of less than 4 h [36]. Overexpression of NMNATs significantly delayed Wallerian degeneration of peripheral nerves [37]. hNMNAT2 was identified as a survival factor for maintaining neuronal health in peripheral nerves. In a mouse model, rTg5410 with a mutation associated with frontotemporal dementia with parkinsonism linked to chromosome 17, NMNAT2 levels were reduced prior to onset of neurodegeration and cognitive deficits. Overexpression of NMNAT2 or NMNAT1 and not NMNAT3 in hippocampi of rTg5410 mice from 6 weeks of age lead to reduced neurodegeration [38].

Since colorectal cancer cells express lower levels of NMNAT compared to leukemia, hepatoma, neuroblastoma, and other cancer cells [9,14,39], it was hypothesized that overexpression of NMNAT in colorectal cancer cell lines would make them more susceptible to tiazofurin. Because hNMNAT2 transfection into cancer cells was done for the first time in this study, it was also important to examine the impact of transfecting NMNAT gene on NAD⁺ levels which is a normal function of NMNAT, and that homeostasis of NAD⁺/NADH determine cellular metabolic functions, oxidative stress and overall cell survival/proliferation. The studies described herein showed that hNMNAT2 transfection into colorectal cancer cells did not perturb NAD⁺ levels. Similar to this observation, an earlier study overexpressing NMNAT1 in yeast showed no alteration in the steady-state level of NAD⁺ [40]. In addition, because NAD⁺ is one of the substrates functioning as an electron acceptor for IMPDH in the pathway for guanylate synthesis and since tiazofurin treatment specifically impedes IMPDH to down-regulate guanylate synthesis, the expression levels of IMPDH in the NMNAT transfected cells was examined. Transfection of hNMNAT2 into colorectal cancer cells did not demonstrate changes in IMPDH expression.

At variance to the instant study is a report that tiazofurin 5′-monophosphate is not a substrate for hNMNAT2 based on in vitro studies conducted with purified human NMNAT isoforms [41]. Those authors expressed the three isoforms of human NMNATs in bacteria and purified proteins were examined for their in vitro catalytic activity to discern organelle selectivity. That study found that tiazofurin 5′-monophosphate exhibited a higher Km as an alternate substrate to NMNAT2 while it exhibited lower mM Km in NMNAT-1 and NMNAT-3. However, the instant studies utilizing intact colorectal cells show that overexpression of hNMNAT2 in colorectal cancer cells results in higher levels of TAD in hNMNAT2-transfected cell lines leading to increased cell-kill with tiazofurin. In addition, there was a good inverse correlation between expression of hNMNAT-2 and cell-kill by tiazofurin in various colorectal cancer cell lines.

Upregulation of NMNAT could benefit tiazofurin treatment because the formation of the active metabolite of tiazofurin, TAD, depends on NMNAT expression and TAD inhibits tumoral IMPDH2, resulting in decrease in GTP pools leading to colorectal cancer cell-kill. Therefore, the influence of hNMNAT2 transfection into colorectal cancer cell lines on the expression of hNMNAT2 and its net effect on tiazofurin cytotoxicity was examined. The results showed that transfection up-regulated hNMNAT2 activity by 2-3-fold, translating to an increase in tiazofurin cell-kill by 2-fold in hNMNAT2 transfected colorectal cancer cell lines. Earlier phase II clinical studies with tiazofurin had shown that peak plasma concentrations of tiazofurin of 240 μM was achievable with a dose of 1,100 mg/m², whereas the starting dose of tiazofurin approved for treatment of patients with chronic myelogenous leukemia in blast crisis is 2,200 mg/m² [9,10,13]. In this study, wild-type and vector transfected Caco2 cells showed an EC50 of about 450 μM; and to produce such a peak plasma concentration of tiazofurin in humans, a dose of 3,300 mg/m² would be required, which could induce increased drug side-effects that interfere with completion of treatment [13]. Therefore, transfection of hNMNAT2 into cancer cells would be helpful for developing targeted treatment for colorectal and other cancers.

These studies also demonstrate that overexpression of hNMNAT2 does not perturb expression of the other two isoforms of hNMNAT, namely hNMNAT1 or hNMNAT3. In addition, silencing of the expression of hNMNAT1 or hNMNAT3 did not change tiazofurin cell-kill either in wild-type or hNMNAT overexpressing colorectal cancer cell lines, suggesting that hNMNAT2 is the likely isoform responsible for activation of tiazofurin in these cell lines. Future studies transfecting or transducing hNMNAT1 or hNMNAT3 into colorectal cancer cells could demonstrate whether these two isoforms can also serve to activate tiazofurin inside cancer cells for therapeutic use.

In summary, here relationship of hNMNAT2 expression with sensitivity to tiazofurin in colorectal cancer cells have been described and it has been shown that transfecting hNMNAT2 increases cell-kill with tiazofurin. Overexpression of hNMNAT2 is confined to colorectal cancer cell cytoplasm and knockdown of hNMNAT2-overexpression with shRNA-hNMNAT2 leads to partial reversal of cell-kill by tiazofurin. Tiazofurin treatment of hNMNAT2-overexpressing cancer cells leads to decrease in IMPDH activity and further lowers GTP concentration. Silencing expression of human NMNAT1 or NMNAT3 isoforms did not alter tiazofurin cell-kill of colorectal cancer cells, indicating that the hNMNAT2 isoform is responsible for tiazofurin action. To increase specificity, FR overexpression in colorectal cancer cells was utilized, and folate-tethered nanoparticles were prepared and showed suitability of targeting FR overexpression. These studies demonstrate the feasibility of developing a targeted gene-directed (hNMNAT2) enzyme pro-drug (tiazofurin) therapy for colorectal cancer. In addition, this study elucidates methods to improve therapeutic efficacy of tiazofurin in colorectal cancer.

### REFERENCES

1. Emanuelli, et al. (2001), J. Biol. Chem. 276, 406-412.
2. Yalowitz, et al. (2004), Biochem. J. 377, 317-326.
3 Raffaelli, et al. (2002), Biochem. Biophys. Res. Commun. 297, 835-840.
4 Zhang, et al. (2003), J. Biol. Chem. 278, 13503-13511.
5 Cooney, et al. (1982), Biochem. Pharmacol. 31, 2133-2136.
6 Jayaram, et al. (1993), Cancer Res. 53, 2344-2348.
7 Zhen, et al. (1992), Cancer Invest. 10, 505-511.
8 Jayaram, et al. (1996), J. Exp. Ther. Oncol. 1, 278-285.
9 Tricot, et al. (1989), Cancer Res. 49, 3696-3701.
10 Wright, et al. (1996), Anticancer Res. 16, 3349-3351.
11 Grifantini, M. (2000), Tiazofurine ICN Pharmaceuticals. Curr. Opin. Investig. Drugs 1, 257-262.
12 Maroun, et al. (1987), Cancer Treat. Rep. 71, 1297-1298.
13 Jayaram, et al. (1992), Int. J. Cancer 51, 182-188.
14 Lapis, et al. (1996), Anticancer Res. 16, 3323-3331.
15 Ahluwalia, et al. (1984), Biochem. Pharmacol. 33, 1195-1203.
16 Jayaram, H. N. (1985), Adv. Enzyme Regul. 24, 67-89.
17 Shia, et al. (2008). Hum. Pathol. 39, 498-505.
18 Kalli, et al. (2008), Gynecol. Oncol. 108, 619-626.
19 Lee and Low (1995), Biochim. Biophys. Acta 1233, 134-144.
20 Zhang, et al. (2004), Anal Biochem. 332, 168-177.
21 Xiang, et al. (2008), Int. J. pharm. 356, 29-36.
22 Suzuki, et al. (2008), Int. J. Pharm. 346, 143-150.
23 Wang, et al. (2010), HSV-TK/GCV cancer suicide gene therapy by a designed recombinant multifunctional vector. Nanomedicine, October 1. doi:10.1016/j.nano.2010.08.003.
24 Zhang, et al. (2010), Cancer Biother. Radiopharm. 25, 487-495. Antony, et al. (2004), J. Clin. Invest. 113, 285-301.
26 Egan, R. W. (1976), J. Biol. Chem. 251, 4442-4447.
27 Gharehbaghi, et al. (1994), Biochem. Pharmacol. 48, 1413-1419.
28 Andre, et al. (2004), N. Engl. J. Med. 350, 2343-2351.
29 Goldberg, et al. (2004), J. Clin. Oncol. 22, 23-30.
30 et al. (2004), Ann. Oncol. 15.1210-1214.
31 Arkenau, et al. (2008), J. Clin. Oncol. 26, 5910-5917.
32 Berger, et al. (2005), J. Biol. Chem. 280, 36334-36341.
33 Mayer, et al. (2010), J. Biol. Chem. 285. 40387-40396.
34 Di Stefano, et al. (2010), Blood Cells Mol. Dis. 45, 33-39.
35 AD; website: www.nextbio.com
36 Sasaki and Milbrandt (2010), PLoS Biol. 285, 41211-5.
37 Gilley and Coleman (2010), PLos Biol. 8, e1000300.
38 Ljungberg, et al. (2012), Hum. Mol. Genet. 21, 251-67.
39 Pillwein, et al. (1993), Int. J. Cancer 55, 92-95.
40 Anderson, et al. (2002), J. Biol. Chem. 277, 18881-18890.
41 Sorci, et al. (2007), Biochemistry 46, 4912-4922.

All of the compositions and methods described and claimed herein can be made and executed without undue experimentation in light of the present disclosure. While the compositions and methods of this invention have been described in terms of preferred embodiments, it will be apparent to those of skill in the art that variations may be applied to the compositions and methods. All such similar substitutes and modifications apparent to those skilled in the art are deemed to be within the spirit and scope of the invention as defined by the appended claims.

The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of treating colorectal cancer, comprising administering to a patient known to have or suspected of having colorectal cancer a composition that comprises a nanoparticle-forming formulation and a chemotherapeutic agent, wherein the patient is also optionally administered a composition that increases intracellular expression of hNMNAT2.

2. A method according to claim 1 wherein the composition that comprises a nanoparticle-forming formulation and a chemotherapeutic agent further comprises a folate receptor (FR) tag, wherein the FR tag optionally is an antibody or antigen-binding antibody fragment that binds a FR extracellular domain or a FR ligand, optionally folic acid.

3. A method according to claim 1 wherein the nanoparticle-forming formulation comprises distearoylphosphatidylcholine (DSPC), cholesterol, and DSPE-PEG-folate (56:40:0.1 v/v)

4. A method according to claim 1 wherein the chemotherapeutic agent is tiazofurin.

5. A method according to claim 1 wherein the composition that increases intracellular expression of hNMNAT2 comprises an expression construct that codes for expression of hNMNAT.

6. A pharmaceutical composition that enhances tiazofurin sensitivity, comprising a pharmaceutically acceptable carrier and (i) an expression construct that codes for expression of hNMNAT and/or (ii) an effective amount of hNMNAT.

7. A method for enhancing colorectal cancer cell tiazofurin sensitivity, comprising administering an amount of a composition according to claim sufficient to increase tiazofurin metabolism in colorectal cancer cells, thereby enhancing colorectal cancer cell tiazofurin sensitivity.

8. A diagnostic method, comprising assessing a level of expression of hNMNAT in a biological sample, optionally a biological sample selected from the group consisting of blood, plasma, serum, an a tissue biopsy, optionally a tissue biopsy known or suspected to contain colorectal cancer cells.

9. A method according to claim 9 that further comprises assessing whether a patient known or suspected to have colorectal cancer is likely to respond to tiazofurin therapy.
